(19) United States
(12) Patent Application Publication
(10) Pub. No.: US 2023/0220349 A1
(43) Pub. Date: Jul. 13, 2023

(54) 3D CULTURE OF MESENCHYMAL LINEAGE PRECURSOR OR STEM CELLS

(71) Applicant: MESOBLAST INTERNATIONAL SÁRL, Meyrin (CH)

(72) Inventor: Paul SIMMONS, Melbourne, Victoria (AU)

(21) Appl. No.: 18/001,462

(22) PCT Filed: Jun. 9, 2021
(86) PCT No.: PCT/IB2021/055055

§ 371 Date: Dec. 9, 2022

(30) Foreign Application Priority Data
Jun. 11, 2020 (AU) 2020901931

(51) Int. Cl. C12N 5/0775

(52) U.S. Cl. CPC C12N 5/0662; C12N 2500/92; C12N 2501/115; C12N 2501/135; C12N 2501/11; C12N 2531/00; C12N 2533/54

(57) ABSTRACT

The present disclosure relates to improved methods serum free stem cell culture, particularly 3D culture in bioreactors as well as cell culture medium and compositions for use in the same. Such methods may be particularly suitable for large scale cell manufacture.

### TECHNICAL FIELD

The present disclosure relates to improved methods serum free stem cell culture, particularly 3D culture in bioreactors as well as cell culture medium and compositions for use in the same.

### BACKGROUND

Multipotent mesenchymal lineage cells (MLC) have been proposed as an attractive candidate for therapeutic applications because of their high proliferation and differentiation potential as well as immunoregulatory and other beneficial properties (Caplan A I (2007) J. Cell Physiol., 213, 341-347; Prockop D J (2007) Clin Pharmacol Ther., 82, 241-243). However, one of the most important and immediate challenges faced is the need to translate the highly individualized in vitro requirements of cellular products into large-scale, streamlined bioprocesses that are reproducible, robust, and safe.

Conventional media used for isolating and expanding MSC consist of a defined basal medium (e.g. Dulbecco's modified Eagle's medium (DMEM) or α-modified minimum essential medium (α-MEM)) supplemented with fetal bovine serum because of its high content of stimulatory growth factors. Although these media are generally reported to support the proliferation of MSC for multiple passages, concerns have been raised because of the potential risks associated with fetal bovine serum (Dimarakis & Levicar (2006) Stem Cells, 24, 1407-1408; Mannello & Tonti (2007) Stem Cells, 25, 1603-1609). In particular, fetal bovine serum may contain harmful contaminants such as prion, viral and zoonotic agents, and can elicit immune reactions. Moreover, the poorly defined nature of fetal bovine serum, and its high degree of batch-to-batch variation, can cause inconsistencies in the growth-supporting properties of media, and thus make standardization of a cell production process difficult.

Human sourced supplements, such as human serum and platelet lysate, have been investigated as a replacement for fetal bovine serum. Human serum is not generally considered a suitable replacement because of its lack of availability and inconsistent growth-promoting potential. Human platelet-derived supplements such as platelet lysate (hPL) and platelet-rich plasma have recently been proposed as a superior alternative (Doucet et al. (2005) J Cell Physiol., 205, 228-236; Muller et al. (2006), Cytotherapy, 8, 437-444; Capelli et al. (2007) Bone Marrow Transplant, 40, 785-91; Lange et al. (2007) J Cell Physiol., 213, 18-26; Reinisch et al. (2007) Regen Med., 2, 371-82). While these studies demonstrated considerable growth-promoting properties of pooled human platelet derivatives, their impact on MLC growth is not consistent (Bieback et al. (2008) Transfus Med Hemother., 35, 286-294). Furthermore, the high cost of these hPL formulations can be prohibitive for commercial cell culture.

Accordingly, there remains an unmet need for cost effective methods of supporting both the isolation and rapid expansion of MSC in fetal bovine serum free cell culture.

### SUMMARY

The present inventors identified that fetal bovine serum is a surprisingly poor stimulus for mesenchymal lineage or stem cell growth in three dimensional (3D) bioreactor culture despite being an effective stimulus for mesenchymal lineage or stem cell growth in a two dimensional (2D) setting. Accordingly, the present inventors noted that growth media effective in a 2D setting may not be effective in a 3D setting. Furthermore, by removing animal serum from culture media, the present inventors identified that other non-animal growth stimuli such as human platelet lysate (hPL) are particularly effective at promoting growth of stem cells in a 3D setting. Accordingly, in an example, the present disclosure relates to a method of culturing mesenchymal lineage precursor or stem cells in a three dimensional culture, the method comprising culturing a population of mesenchymal lineage precursor or stem cells in a cell culture medium, wherein the cell culture medium is animal serum free.

hPL comprises various growth factors such as platelet derived growth factor (PDGF), fibroblast growth factor 2 (FGF2) and epidermal growth factor (EGF). The present inventors identified the importance of PDGF and FGF2 in promoting mesenchymal lineage or stem cells in a 3D setting. Accordingly, in an example, the culture medium is animal serum free and comprises PDGF and FGF2. In another example, the culture medium further comprises EGF. In an example, the mesenchymal lineage precursor or stem cells are cultured in a bioreactor.

The present inventors also identified that culturing mesenchymal lineage or stem cells in 3D culture on an adherent material was important for growth in a 3D setting when the culture medium was animal serum free. Accordingly, in another example, the present disclosure relates to a method of culturing mesenchymal lineage precursor or stem cells in a three dimensional culture, the method comprising culturing a population of mesenchymal lineage precursor or stem cells on an adherent material in a cell culture medium, wherein the mesenchymal lineage precursor or stem cells are attached to the adherent material and, wherein the cell culture medium is animal serum free. In an example, the culture medium further comprises PDGF and FGF2. In another example, the culture medium further comprises EGF. In an example, the mesenchymal lineage precursor or stem cells are cultured in a bioreactor. In an example, the adherent material is a microcarrier.

The present inventors subsequently identified that culturing mesenchymal lineage precursor or stem cells on certain adherent materials in 3D culture is problematic as viable cell numbers drop significantly at or around peak cell density. Surprisingly, this problem was mitigated by culturing mesenchymal lineage or stem cells on certain adherent materials, particularly degradable microcarriers such as those with degradable cores and/or microcarriers of low density. Accordingly, in an example, the microcarrier is degradable. In another example, the microcarrier has a degradable core. In another example, the microcarrier has a carbohydrate polymer or glycoprotein core.

In various examples, the adherent material such as a microcarrier can be coated. In an example, the adherent material is coated. In another example, the microcarrier is coated. In an example, the adherent material or microcarrier is coated with a glycoprotein. In an example, the glycoprotein is a collagen or vitronectin. In an example, the vitronectin is human vitronectin or a synthetic mimetic thereof. Synthetic mimetics of vitronectin are capable of binding and supporting growth of mesenchymal lineage precursor or stem cells on their surface. In another example, the glycoprotein is synthetic. Accordingly, in an example, the present disclosure encompasses 3D cell culture which comprises culturing a population of mesenchymal lineage precursor or stem cells on an adherent material in a cell culture medium, wherein the mesenchymal lineage precursor or stem cells are attached to the adherent material, wherein the cell culture medium is animal serum free, and, wherein the adherent material is coated with a glycoprotein such as a nectin or a collagen.

In another example, the microcarrier comprises a carbohydrate polymer core, wherein the carbohydrate polymer is linked in a calcium dependent manner.

In an example, the microcarrier has a density of about 0.5 to 5 g/ml. In another example, the microcarrier has a density of about 0.5 to 3 g/ml. In an example, the culture medium comprises 0.5 g/L to 5 g/L of microcarrier. In another example, the culture medium comprises 0.5 g/L to 3 g/L of microcarrier. In another example, the culture medium comprises 0.5 g/L to 2 g/L of microcarrier. In another example, the culture medium comprises about 1 g/L of microcarrier.

In an example, the microcarrier is porous. In another example, the microcarrier is macroporous.

In an example, the cell culture medium is free of animal components.

The present inventors also surprisingly found that replacing a specified amount of medium every 24 hours in bioreactor culture was associated with improvements in cell growth. Accordingly, in an example, the methods of the present disclosure comprise replacing between 60 and 80% of medium every 24 hours of culture. In another example, the methods of the present disclosure comprise replacing about 70% of medium every 24 hours of culture. In these examples, media replacement may begin from day 2 to 4 of culture in a bioreactor. In an example, media replacement begins from day 3 of culture in a bioreactor.

In an example, the methods of the disclosure further comprise dissociating the mesenchymal lineage precursor or stem cells from the adherent material by contacting them with a dissociating agent. In an example, mesenchymal lineage precursor or stem cells are dissociated from the adherent material after reaching peak cell density. In an example, mesenchymal lineage precursor or stem cells are dissociated after about 7 days of culture in a bioreactor.

In an example, the methods of the disclosure further comprise degrading the adherent material or microcarrier. In an example, the adherent material or microcarrier is degraded by adding an enzyme to the culture medium. In an example, the microcarrier comprises vitronectin, for example, is coated in vitronectin and the enzyme is recombinant pectinase. In this example, the microcarrier may comprise a carbohydrate core linked in a calcium dependent manner in which case, EDTA and an enzyme such as recombinant pectinase may be added to the culture medium.

In an example, mesenchymal lineage precursor or stem cells are seeded in 3D culture at between 5,000 and 20,000 cells/ml. In another example, mesenchymal lineage precursor or stem cells are seeded at 10,000 cells/ml.

In an example, mesenchymal lineage precursor or stem cells have been culture expanded from a master cell bank. In an example, the mesenchymal lineage precursor or stem cells have been culture expanded from a master cell bank in a two dimensional culture format.

In another example, the methods of the disclosure further comprise recovering the cells from the culture medium and cryopreserving the recovered cells. In an example, recovered cells are washed and concentrated prior to cryopreservation.

In an example, the mesenchymal lineage precursor or stem cells are cultured in a three dimensional culture for at least 6 days, preferably between 5 and 8 days, more preferably 7 days.

In an example, the bioreactor is a stirred tank bioreactor. In another example, the bioreactor is a packed bed bioreactor. In another example, the bioreactor is a stirred tank and/or packed bed bioreactor.

In another example, the present disclosure encompasses a composition comprising a population of mesenchymal lineage precursor or stem cells and cell culture medium, wherein the cell culture medium is animal serum free and comprises an adherent material, PDGF and FGF2, and wherein the mesenchymal lineage precursor or stem cells are attached to the adherent material. In an example, the adherent material is as defined above. For example, the adherent material may be a microcarrier.

In an example, the mesenchymal lineage precursor or stem cells are mesenchymal precursor cells or mesenchymal stem cells. In an example, the mesenchymal lineage precursor or stem cells are mesenchymal precursor cells. In an example, the mesenchymal lineage precursor or stem cells are mesenchymal stem cells.

In an example, the PDGF in the culture medium is PDGF-BB. In an example, the culture medium comprises between 3.0 ng/ml and 120 ng/ml of PDGF-BB. In another example, the culture medium comprises between 2 pg/ml and 6 ng/ml of FGF2. In another example, the culture medium comprises less than 0.8 ng/ml of FGF2. In another example, the culture medium further comprises EGF. In another example, the culture medium further comprises between 0.08 ng/ml and 7 ng/ml of EGF.

In an example, the culture medium comprises alpha-minimal essential medium or fetal bovine serum free expansion medium. In an example, the culture medium is serum free. In an example, the culture medium maintains the stem cells in an undifferentiated state.

In another example, the present disclosure relates to a method of culturing stem cells in a bioreactor, the method comprising culturing a population of mesenchymal lineage precursor or stem cells in a bioreactor comprising cell culture medium, wherein the cell culture medium is animal serum free and comprises platelet derived growth factor (PDGF) and fibroblast growth factor 2 (FGF2) and optionally EGF. Accordingly, in this example, the culture medium may comprise EGF.

### BRIEF DESCRIPTION OF DRAWINGS

FIG. 1: Proliferation and maximum cell densities following mesenchymal lineage cell (MLC) culture with EMD-Millipore using medium supplemented with hPL at the stated concentrations. The outcomes of previous runs conducted with the same MLC bank in fetal bovine serum are shown for comparison.

FIG. 2: V2.2 supports robust proliferation of MLC in the Millipore Mobius 50 L Cell Ready bioreactor.

FIG. 3: MLC from different donors exhibit different proliferation kinetics and yields in V2.2.

FIG. 4: The media exchange/harvest strategy used to prepare seed has no impact on the subsequent yields generated in V2.2 in spinner flasks.

FIG. 5: Harvesting CF10 cell factories seeded with a single MCB from 8 different donors at Day 6 following a media exchange at Day 4 (D4MX/D6H) generates the target number of 400 million cells for 7/8 MCBs.

FIG. 6: Comparison of the performance of Cultispher-G microcarriers vs. Solohill collagen coated microcarriers in the BioBLU 3c in V2.2 medium.

FIG. 7: Use of Cultispher-G microcarriers with the BioBLU 50c single use BioR results in a highly reproducible yield of MLC driven by V2.2 and a stable plateau after peak numbers are achieved.

FIG. 8: Comparison of the performance in spinner flasks of Cultispher G, Solohill collagen-coated microcarriers with that of the Corning DMC coated with either collagen or Synthemax.

FIG. 9: Comparison of the yields obtained in spinner flasks (100 mL volume) using the Corning Synthemax DMC across different MLC banks.

FIG. 10: Highly reproducible yields of MLC in the BioBLU 50c using V2.2 and Corning Synthemax microcarriers. Yields are not influenced by regulation of DO

FIG. 11: Post-thaw viability of MLC from MCB019 following propagation in V2.2 on Corning Synthemax DMC in the BioBLU 50c bioreactor. Those produced in the absence of dissolved oxygen (DO) control-blue; Those with DO control-red.

FIG. 12: Immediate post thaw cell diameter of cryopreserved MLC generated following propagation in V2.2 on Corning Synthemax DMC in the BioBLU 50c bioreactor. Those produced in the absence of DO control-blue; Those with DO control-red.

FIG. 13: Post-thaw proliferation kinetics of cryopreserved MLC generated in V2.2 on Corning Synthemax DMC in the BioBLU 50c bioreactor.

FIG. 14: Average cell numbers per well at Day 6 generated by cryopreserved MLC generated in V2.2 on Corning Synthemax DMC in the BioBLU 50c bioreactor. Those produced in the absence of DO control-blue; Those with DO control-red.

FIG. 15: Flow cytometric analysis of MLC identity (upper panel) and of MLC purity marker expression (lower panel) on cryopreserved MLC post-thaw generated in V2.2 on Corning Synthemax DMC in the BioBLU 50c bioreactor.

FIG. 16: SDF-1α levels in conditioned medium of MLC generated in V2.2 on Corning Synthemax DMC in the BioBLU 50c bioreactor.

FIG. 17: SDF-1α bioactivity as measured using an SDF-1α dependent migration assay in conditioned medium from MLC generated in V2.2 on Corning Synthemax DMC in the BioBLU 50c bioreactor.

FIG. 18: VEGF-A levels in conditioned medium of MLC generated in V2.2 on Corning Synthemax DMC in the BioBLU 50c bioreactor.

FIG. 19: ANGPT1 levels in conditioned medium of MLC generated in V2.2 on Corning Synthemax DMC in the BioBLU 50c bioreactor.

FIG. 20: MLC generated in V2.2 on Corning Synthemax DMC in the BioBLU 50c bioreactor consistently demonstrate a potent ability to inhibit proliferation of activated allogeneic T-cells.

FIG. 21: Schematic overview of the bioreactor process for MLC manufacture.

### DETAILED DESCRIPTION

### General Techniques and Definitions

Unless specifically defined otherwise, all technical and scientific terms used herein shall be taken to have the same meaning as commonly understood by one of ordinary skill in the art (e.g., in cell culture, molecular biology, stem cell culture, immunology, and biochemistry).

Unless otherwise indicated, cell culture techniques and assays utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, refers to +/−10%, more preferably +/−5%, of the designated value.

The term “level” is used to define the amount of a particular substance present in the cell culture medium and compositions of the present disclosure. For example, a particular concentration, weight, percentage (e.g. v/v %) or ratio can be used to define the level of a particular substance.

The term “sufficient” is used herein to define an amount of growth factor that provides a specific concentration when dissolved in a stem cell culture medium. In this context, a “sufficient amount” is dictated by the volume of culture medium required. For example, if the required concentration of FGF2 in a stem cell culture medium was about 10 pg/ml and 500 ml of cell culture media was required, a sufficient amount would be about 5 ng.

In the context of releasing mesenchymal lineage precursor or stem cells from adherent material, the term “sufficient” is used to refer to vibration for a period of time and at a frequency and amplitude that is sufficient to release the mesenchymal lineage precursor or stem cells from the adherent material.

The term “seeding” is used herein to refer to the process of introducing cells into 3 dimensional (3D) culture. In an example, the methods of the disclosure encompass dynamic seeding, wherein the culture medium continues to mix as cells attach to adherent material. In an another example, the cells are seeded into 3D culture and left for a period of time sufficient to adhere to the adherent material in the culture medium, such that the cells can attach to the material. In some embodiments, the step of seeding the cells into the bioreactor is effected while flow in the bioreactor is shut off for at least 10 hours following the seeding.

In an example, mesenchymal lineage precursor or stem cells are seeded at between 5,000 and 20,000 cells/ml. In another example, mesenchymal lineage precursor or stem cells are seeded at between 8,000 and 20,000 cells/ml. In another example, mesenchymal lineage precursor or stem cells are seeded at between 8,000 and 15,000 cells/ml. In another example, mesenchymal lineage precursor or stem cells are seeded at 5,000 cells/ml. In another example, mesenchymal lineage precursor or stem cells are seeded at 8,000 cells/ml. In another example, mesenchymal lineage precursor or stem cells are seeded at least at 8,000 cells/ml. In another example, mesenchymal lineage precursor or stem cells are seeded at 10,000 cells/ml.

The term “recovering” is used herein to refer to removing cells from 2D or 3D culture. For example, cells can be recovered from a bioreactor culture disclosed herein. In an example, recovered cells are first washed (e.g. 2-3 times) with a saline solution or comparable solution. Subsequent to the washing step, a dissociating step may be conducted on the adherent material. In one example, a suitable dissociation enzyme is employed during the dissociating step. In an example, cells recovered from 3D culture are washed and concentrated before being cryopreserved. In an example, the washed and concentrated cells can be stored, filled, finished and visually inspected before being cryopreserved.

As used herein, a “dissociating agent” is any compound that serves to disrupt points of attachment between a cell and a surface to which the cell is attached. In some embodiments, the dissociating agent is an enzyme. In particular embodiments, the enzyme is trypsin, including recombinant trypsin, papain, elastase, hyaluronidase, collagenase type 1, collagenase type 2, collagenase type 3, collagenase type 4, or dispase. In an example, the dissociating agent comprises EDTA. In an example the dissociating agent comprises EDTA and an enzyme. For example, the dissociating agent may comprise EDTA and pectinase. In an example, the dissociating agent may comprise EDTA and a collagenase. Those of skill in the art will appreciate that EDTA may be an appropriate dissociating agent for microcarriers with a carbohydrate core linked in a calcium dependent manner. In an example, the dissociating agent also degrades the microcarrier. For example, the dissociating agent can degrade the microcarrier core.

In an example, cells can be dissociated from adherent material disclosed herein using a dissociating agent. For example, the dissociating agent can be fed into a bioreactor disclosed herein to dissociate cells as required. In an example, cell culture may be filtered or partially filtered before being contacting with an appropriate dissociating agent. For example, the cells may be contacted with a dissociating agent after reaching peak cell density. In another example, cultured cells can be dissociated from adherent material via vibration. “Vibration” means mechanical oscillations about an equilibrium point. The oscillations may be periodic or random. In one embodiment, the vibration is due to reciprocating linear oscillations that are controlled with respect to amplitude and frequency. In some embodiments, the amplitude and frequency of the oscillations are constant, while in other embodiments either or both of the amplitude or frequency may be varied as desired to achieve dissociation of cells. Other examples, the duration of the period of time for the vibrations is also controlled using means and devices that are conventional in the art. In some examples, vibration is provided by an electro-mechanical device, for example, an electric motor with an unbalanced mass on its driveshaft. In other examples, vibration is provided by an electrical device. Various examples, of devices capable of imparting vibrations are known in the art.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally-equivalent products, compositions and methods are clearly within the scope of the disclosure, as described herein.

Any example disclosed herein shall be taken to apply mutatis mutandis to any other example unless specifically stated otherwise.

### Mesenchymal Lineage Precursor Cells

As used herein, the term “mesenchymal lineage precursor or stem cell (MLPSC)” refers to undifferentiated multipotent cells that have the capacity to self-renew while maintaining multipotency and the capacity to differentiate into a number of cell types either of mesenchymal origin, for example, osteoblasts, chondrocytes, adipocytes, stromal cells, fibroblasts and tendons, or non-mesodermal origin, for example, hepatocytes, neural cells and epithelial cells. For the avoidance of doubt, a “mesenchymal lineage precursor cell” refers to a cell which can differentiate into a mesenchymal cell such as bone, cartilage, muscle and fat cells, and fibrous connective tissue.

The term “mesenchymal lineage precursor or stem cells” includes both parent cells and their undifferentiated progeny. The term also includes mesenchymal precursor cells, multipotent stromal cells, mesenchymal stem cells (MSCs), perivascular mesenchymal precursor cells, and their undifferentiated progeny.

Mesenchymal lineage precursor or stem cells can be autologous, allogeneic, xenogenic, syngenic or isogenic. Autologous cells are isolated from the same individual to which they will be reimplanted. Allogeneic cells are isolated from a donor of the same species. Xenogenic cells are isolated from a donor of another species. Syngenic or isogenic cells are isolated from genetically identical organisms, such as twins, clones, or highly inbred research animal models.

In an example, the mesenchymal lineage precursor or stem cells are allogeneic. In an example, the allogeneic mesenchymal lineage precursor or stem cells are culture expanded and cryopreserved.

Mesenchymal lineage precursor or stem cells reside primarily in the bone marrow, but have also shown to be present in diverse host tissues including, for example, cord blood and umbilical cord, adult peripheral blood, adipose tissue, trabecular bone and dental pulp. They are also found in skin, spleen, pancreas, brain, kidney, liver, heart, retina, brain, hair follicles, intestine, lung, lymph node, thymus, ligament, tendon, skeletal muscle, dermis, and periosteum; and are capable of differentiating into germ lines such as mesoderm and/or endoderm and/or ectoderm. Thus, mesenchymal lineage precursor or stem cells are capable of differentiating into a large number of cell types including, but not limited to, adipose, osseous, cartilaginous, elastic, muscular, and fibrous connective tissues. The specific lineage-commitment and differentiation pathway which these cells enter depends upon various influences from mechanical influences and/or endogenous bioactive factors, such as growth factors, cytokines, and/or local microenvironmental conditions established by host tissues.

The terms “enriched”, “enrichment” or variations thereof are used herein to describe a population of cells in which the proportion of one particular cell type or the proportion of a number of particular cell types is increased when compared with an untreated population of the cells (e.g., cells in their native environment). In one example, a population enriched for mesenchymal lineage precursor or stem cells comprises at least about 0.1% or 0.5% or 1% or 2% or 5% or 10% or 15% or 20% or 25% or 30% or 50% or 75% mesenchymal lineage precursor or stem cells. In this regard, the term “population of cells enriched for mesenchymal lineage precursor or stem cells” will be taken to provide explicit support for the term “population of cells comprising X % mesenchymal lineage precursor or stem cells”, wherein X % is a percentage as recited herein. The mesenchymal lineage precursor or stem cells can, in some examples, form clonogenic colonies, e.g. CFU-F (fibroblasts) or a subset thereof (e.g., 50% or 60% or 70% or 70% or 90% or 95%) can have this activity.

In an example of the present disclosure, the mesenchymal lineage precursor or stem cells are mesenchymal stem cells (MSCs). The MSCs may be a homogeneous composition or may be a mixed cell population enriched in MSCs. Homogeneous MSC compositions may be obtained by culturing adherent marrow or periosteal cells, and the MSCs may be identified by specific cell surface markers which are identified with unique monoclonal antibodies. A method for obtaining a cell population enriched in MSCs is described, for example, in U.S. Pat. No. 5,486,359. Alternative sources for MSCs include, but are not limited to, blood, skin, cord blood, muscle, fat, bone, and perichondrium. In an example, the MSCs are allogeneic. In an example, the MSCs are cryopreserved. In an example, the MSCs are culture expanded and cryopreserved.

In another example, the mesenchymal lineage precursor or stem cells are CD29+, CD54+, CD73+, CD90+, CD102+, CD105+, CD106+, CD166+, MHC1+ MSCs.

Isolated or enriched mesenchymal lineage precursor or stem cells can be expanded in vitro by culture. Isolated or enriched mesenchymal lineage precursor or stem cells can be cryopreserved, thawed and subsequently expanded in vitro by culture.

In one example, isolated or enriched mesenchymal lineage precursor or stem cells are seeded at 50,000 viable cells/cm² in culture medium (serum free or serum-supplemented), for example, alpha minimum essential media (αMEM) supplemented with 5% fetal bovine serum (FBS) and glutamine, and allowed to adhere to the culture vessel overnight at 37° C., 20% O₂. The culture medium is subsequently replaced and/or altered as required and the cells cultured for a further 68 to 72 hours at 37° C., 5% O₂.

As will be appreciated by those of skill in the art, cultured mesenchymal lineage precursor or stem cells are phenotypically different to cells in vivo. For example, in one embodiment they express one or more of the following markers, CD44, NG2, DC146 and CD140b. Cultured mesenchymal lineage precursor or stem cells are also biologically different to cells in vivo, having a higher rate of proliferation compared to the largely non-cycling (quiescent) cells in vivo.

In one example, the population of cells is enriched from a cell preparation comprising STRO-1+ cells in a selectable form. In this regard, the term “selectable form” will be understood to mean that the cells express a marker (e.g., a cell surface marker) permitting selection of the STRO-1+ cells. The marker can be STRO-1, but need not be. For example, as described and/or exemplified herein, cells (e.g., mesenchymal precursor cells) expressing STRO-2 and/or STRO-3 (TNAP) and/or STRO-4 and/or VCAM-1 and/or CD146 and/or 3G5 also express STRO-1 (and can be STRO-1bright). Accordingly, an indication that cells are STRO-1+ does not mean that the cells are selected solely by STRO-1 expression. In one example, the cells are selected based on at least STRO-3 expression, e.g., they are STRO-3+ (TNAP+).

Reference to selection of a cell or population thereof does not necessarily require selection from a specific tissue source. As described herein STRO-1+ cells can be selected from or isolated from or enriched from a large variety of sources. That said, in some examples, these terms provide support for selection from any tissue comprising STRO-1+ cells (e.g., mesenchymal precursor cells) or vascularized tissue or tissue comprising pericytes (e.g., STRO-1+ pericytes) or any one or more of the tissues recited herein.

In one example, the cells used in the present disclosure express one or more markers individually or collectively selected from the group consisting of TNAP+, VCAM-1+, THY-1+, STRO-2+, STRO-4+(HSP-90β), CD45+, CD146+, 3G5+ or any combination thereof.

By “individually” is meant that the disclosure encompasses the recited markers or groups of markers separately, and that, notwithstanding that individual markers or groups of markers may not be separately listed herein the accompanying claims may define such marker or groups of markers separately and divisibly from each other.

By “collectively” is meant that the disclosure encompasses any number or combination of the recited markers or groups of markers, and that, notwithstanding that such numbers or combinations of markers or groups of markers may not be specifically listed herein the accompanying claims may define such combinations or sub-combinations separately and divisibly from any other combination of markers or groups of markers.

As used herein the term “TNAP” is intended to encompass all isoforms of tissue non-specific alkaline phosphatase. For example, the term encompasses the liver isoform (LAP), the bone isoform (BAP) and the kidney isoform (KAP). In one example, the TNAP is BAP. In one example, TNAP as used herein refers to a molecule which can bind the STRO-3 antibody produced by the hybridoma cell line deposited with ATCC on 19 Dec. 2005 under the provisions of the Budapest Treaty under deposit accession number PTA-7282.

Furthermore, in one example, the STRO-1+ cells are capable of giving rise to clonogenic CFU-F.

In one example, a significant proportion of the STRO-1+ cells are capable of differentiation into at least two different germ lines. Non-limiting examples of the lineages to which the STRO-1+ cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells.

In an example, mesenchymal lineage precursor or stem cells are obtained from a single donor, or multiple donors where the donor samples or mesenchymal lineage precursor or stem cells are subsequently pooled and then culture expanded.

Mesenchymal lineage precursor or stem cells encompassed by the present disclosure may also be cryopreserved prior to administration to a subject. In an example, mesenchymal lineage precursor or stem cells are culture expanded and cryopreserved prior to administration to a subject.

In an example, the present disclosure encompasses mesenchymal lineage precursor or stem cells as well as progeny thereof, soluble factors derived therefrom, and/or extracellular vesicles isolated therefrom. In another example, the present disclosure encompasses mesenchymal lineage precursor or stem cells as well as extracellular vesicles isolated therefrom. For example, it is possible to culture expand mesenchymal precursor lineage or stem cells of the disclosure for a period of time and under conditions suitable for secretion of extracellular vesicles into the cell culture medium. Secreted extracellular vesicles can subsequently be obtained from the culture medium for use in therapy.

The term “extracellular vesicles” as used herein, refers to lipid particles naturally released from cells and ranging in size from about 30 nm to as a large as 10 microns, although typically they are less than 200 nm in size. They can contain proteins, nucleic acids, lipids, metabolites, or organelles from the releasing cells (e.g., mesenchymal stem cells; STRO-1⁺ cells).

The term “exosomes” as used herein, refers to a type of extracellular vesicle generally ranging in size from about 30 nm to about 150 nm and originating in the endosomal compartment of mammalian cells from which they are trafficked to the cell membrane and released. They may contain nucleic acids (e.g., RNA; microRNAs), proteins, lipids, and metabolites and function in intercellular communication by being secreted from one cell and taken up by other cells to deliver their cargo.

### Culture Expansion of the Cells

In an example, mesenchymal lineage precursor or stem cells are culture expanded. “Culture expanded” mesenchymal lineage precursor or stem cells media are distinguished from freshly isolated cells in that they have been cultured in cell culture medium and passaged (i.e. sub-cultured). In an example, culture expanded mesenchymal lineage precursor or stem cells are culture expanded for about 4-10 passages. In an example, mesenchymal lineage precursor or stem cells are culture expanded for at least 5, at least 6, at least 7, at least 8, at least 9, at least 10 passages. For example, mesenchymal lineage precursor or stem cells can be culture expanded for at least 5 passages. In an example, mesenchymal lineage precursor or stem cells can be culture expanded for at least 5-10 passages. In an example, mesenchymal lineage precursor or stem cells can be culture expanded for at least 5-8 passages. In an example, mesenchymal lineage precursor or stem cells can be culture expanded for at least 5-7 passages. In an example, mesenchymal lineage precursor or stem cells can be culture expanded for more than 10 passages. In another example, mesenchymal lineage precursor or stem cells can be culture expanded for more than 7 passages. In these examples, stem cells may be culture expanded before being cryopreserved to provide an intermediate cryopreserved MLPSC population. In an example, methods of the present disclosure culture cells from an intermediate cryopreserved MLPSC population.

In an embodiment, mesenchymal lineage precursor or stem cells can be obtained from a single donor, or multiple donors where the donor samples or mesenchymal lineage precursor or stem cells are subsequently pooled and then culture expanded. In an example, the culture expansion process comprises:

i. expanding by passage expansion the number of viable cells to provide a preparation of at least about 1 billion of the viable cells, wherein the passage expansion comprises establishing a primary culture of isolated mesenchymal lineage precursor or stem cells and then serially establishing a first non-primary (P1) culture of isolated mesenchymal lineage precursor or stem cells from the previous culture;

ii. expanding by passage expansion the P1 culture of isolated mesenchymal lineage precursor or stem cells to a second non-primary (P2) culture of mesenchymal lineage precursor or stem cells; and,

iii. preparing and cryopreserving an in-process intermediate mesenchymal lineage precursor or stem cells preparation obtained from the P2 culture of mesenchymal lineage precursor or stem cells; and,

iv. thawing the cryopreserved in-process intermediate mesenchymal lineage precursor or stem cells preparation and expanding by passage expansion the in-process intermediate mesenchymal lineage precursor or stem cells preparation.

In an example, the expanded mesenchymal lineage precursor or stem cell preparation has an antigen profile and an activity profile comprising:

i. less than about 0.75% CD45+ cells;

ii. at least about 95% CD105+ cells;

iii. at least about 95% CD166+ cells.

In an example, the expanded mesenchymal lineage precursor or stem cell preparation is capable of inhibiting IL2Ra expression by CD3/CD28-activated PBMCs by at least about 30% relative to a control.

In an example, culture expanded mesenchymal lineage precursor or stem cells are culture expanded for about 4-10 passages, wherein the mesenchymal lineage precursor or stem cells have been cryopreserved after at least 2 or 3 passages before being further culture expanded. In an example, mesenchymal lineage precursor or stem cells are culture expanded for at least 1, at least 2, at least 3, at least 4, at least 5 passages, cryopreserved and then further culture expanded for at least 1, at least 2, at least 3, at least 4, at least 5 passages before being cultured according to the methods of the disclosure.

The process of mesenchymal lineage precursor or stem cell isolation and ex vivo expansion can be performed using any equipment and cell handing methods known in the art. Various culture expansion embodiments of the present disclosure employ steps that require manipulation of cells, for example, steps of seeding, feeding, dissociating an adherent culture, or washing. Any step of manipulating cells has the potential to insult the cells. Although mesenchymal lineage precursor or stem cells can generally withstand a certain amount of insult during preparation, cells are preferably manipulated by handling procedures and/or equipment that adequately performs the given step(s) while minimizing insult to the cells.

In an example, mesenchymal lineage precursor or stem cells are washed in an apparatus that includes a cell source bag, a wash solution bag, a recirculation wash bag, a spinning membrane filter having inlet and outlet ports, a filtrate bag, a mixing zone, an end product bag for the washed cells, and appropriate tubing, for example, as described in U.S. Pat. No. 6,251,295, which is hereby incorporated by reference.

In an example, a mesenchymal lineage precursor or stem cell composition cultured according to the present disclosure is 95% homogeneous with respect to being CD105 positive and CD166 positive and being CD45 negative. In an example, this homogeneity persists through ex vivo expansion; i.e. though multiple population doublings.

In an example, mesenchymal lineage precursor or stem cells of the disclosure are culture expanded in 2D culture prior to 3D culture. In an example, mesenchymal lineage precursor or stem cells of the disclosure are culture expanded from a master cell bank. In an example, mesenchymal lineage precursor or stem cells of the disclosure are culture expanded from a master cell bank in 2D culture before seeding in 3D culture. In an example, mesenchymal lineage precursor or stem cells of the disclosure are culture expanded from a master cell bank in 2D culture for at least 3 days before seeding in 3D culture in a bioreactor. In an example, mesenchymal lineage precursor or stem cells of the disclosure are culture expanded from a master cell bank in 2D culture for at least 4 days before seeding in 3D culture in a bioreactor. In an example, mesenchymal lineage precursor or stem cells of the disclosure are culture expanded from a master cell bank in 2D culture for between 3 and 5 days before seeding in 3D culture in a bioreactor. In these examples, 2D culture can be performed in a cell factory. Various cell factory products are available commercially (e.g. Thermofisher, Sigma).

### Modification of the Cells

The mesenchymal lineage precursor or stem cells cultured according to the present disclosure may be altered in such a way that upon administration, lysis of the cell is inhibited. Alteration of an antigen can induce immunological non-responsiveness or tolerance, thereby preventing the induction of the effector phases of an immune response (e.g., cytotoxic T cell generation, antibody production etc.) which are ultimately responsible for rejection of foreign cells in a normal immune response. Antigens that can be altered to achieve this goal include, for example, MHC class I antigens, MHC class II antigens, LFA-3 and ICAM-1.

The mesenchymal lineage precursor or stem cells may also be genetically modified to express proteins of importance for the differentiation and/or maintenance of striated skeletal muscle cells. Exemplary proteins include growth factors (TGF-β, insulin-like growth factor 1 (IGF-1), FGF), myogenic factors (e.g. myoD, myogenin, myogenic factor 5 (Myf5), myogenic regulatory factor (MRF)), transcription factors (e.g. GATA-4), cytokines (e.g. cardiotropin-1), members of the neuregulin family (e.g. neuregulin 1, 2 and 3) and homeobox genes (e.g. Csx, tinman and NKx family).

### Cell Culture Medium

The methods of the present disclosure use fetal bovine serum free stem cell culture medium comprising growth factors that promote mesenchymal lineage precursor or stem cell proliferation. In an embodiment, the culture medium is serum free stem cell culture medium. In an example, in addition to the adherent material discussed below, the cell culture medium used in the methods of the present disclosure comprises:

a basal medium;

platelet derived growth factor (PDGF);

fibroblast growth factor 2 (FGF2).

The term “medium” or “media” as used in the context of the present disclosure, includes the components of the environment surrounding the cells. The media contributes to and/or provides the conditions suitable to allow cells to grow. Media may be solid, liquid, gaseous or a mixture of phases and materials. Media can include liquid growth media as well as liquid media that do not sustain cell growth. Media also include gelatinous media such as agar, agarose, gelatin and collagen matrices. Exemplary gaseous media include the gaseous phase that cells growing on a petri dish or other solid or semisolid support are exposed to. The term “medium” also refers to material that is intended for use in a cell culture, even if it has not yet been contacted with cells.

The culture media of the present disclosure can be prepared by using a basal culture medium. In the context of the present disclosure, “basal culture medium” refers to an unsupplemented medium which is suitable for exposure to cells, for example mesenchymal precursor lineage or stem cells. Basal culture medium includes, for example, Eagles minimal essential (MEM) culture media, alpha modified MEM culture media, StemSpan™ and mixed culture media thereof, and is not particularly restricted providing it can be used for culturing of stem cells.

Further, the cell culture medium of the present disclosure can contain any components such as fatty acids or lipids, vitamins, cytokines, antioxidants, buffering agents, inorganic salts and the like.

The cell culture media used in the present disclosure contains all essential amino acids and may also contain non-essential amino acids. In general, amino acids are classified into essential amino acids (Thr, Met, Val, Leu, Ile, Phe, Trp, Lys, His) and non-essential amino acids (Gly, Ala, Ser, Cys, Gln, Asn, Asp, Tyr, Arg, Pro).

Those of skill in the art will appreciate that for optimal results, the basal medium must be appropriate for the cell line of interest with key nutrients available at adequate levels to enhance cell proliferation. For example, it may be necessary to increase the level of glucose (or other energy source) in the basal medium, or to add glucose (or other energy source) during the course of culture, if this energy source is found to be depleted and to thus limit cell proliferation. In an example, dissolved oxygen (DO) levels can also be controlled.

In an example, the cell culture medium of the present disclosure contains human derived additives. For example, human serum and human platelet cell lysate can be added to the cell culture media used in the methods of the present disclosure.

In an example, the cell culture medium of the present disclosure contains only human derived additives. Thus, in an example, the cell culture media is xeno-free. For avoidance of doubt, in these examples, the culture medium is free of animal proteins. In an example, cell culture medium used in the methods of the disclosure is free of animal components.

### Ascorbic Acid

Ascorbic acid is an essential supplement for the growth and differentiation of various kinds of cells in culture. It is now understood that particular ascorbic acid derivatives are “short acting” because they are not stable in solution, especially under the normal cell culture conditions of neutral pH and 37° C. These short acting derivatives rapidly oxidise into oxalic acid or threonic acid. In culture media (pH 7) at 37° C., oxidation decreases the level of these short acting ascorbic acid derivatives by approximately 80-90% in 24 hours. Accordingly, short acting ascorbic acid derivatives have been replaced with more stable “long acting” ascorbic acid derivatives in conventional cell culture of various cell types.

In the context of the present disclosure the term “short acting” encompasses ascorbic acid derivatives that are oxidised by approximately 80-90% following 24 hours of cell culture under culture conditions of neutral pH and 37° C. In one example, the short acting L-ascorbic acid derivative is a L-ascorbic acid salt. For example, in the context of the present disclosure, L-ascorbic acid sodium salt is a “short acting” ascorbic acid derivative.

In contrast, the term “long acting” encompasses ascorbic acid derivatives that are not oxidised by approximately 80-90% following 24 hours of cell culture under culture conditions of neutral pH and 37° C. In one example, in the context of the present disclosure, L-ascorbic acid-2-phosphate is a “long acting” ascorbic acid derivative. Other examples of long acting ascorbic acid derivatives include Tetrahexyldecyl Ascorbate Magnesium Ascorbyl Phosphate and 2-O-α-D-Glucopyranosyl-L-ascorbic acid. The cell culture medium of the present disclosure can contain short acting ascorbic acid derivatives, long acting ascorbic acid derivatives or mixtures thereof.

### Mitogenic Factors

PDGF and FGF2 synergistically promote stem cell proliferation in in-vitro fetal bovine serum free cell culture.

PDGF is a regulator of cell growth and division which binds to platelet derived growth factor receptors (PDGFR). In chemical terms, PDGF is a dimeric glycoprotein composed of two A (-AA) or two B (-BB) chains or a combination of the two (-AB). PDGF-AB has been shown to bind PDGF alpha and beta receptor subunits to form PDGF alpha beta and alpha alpha receptor dimers. In the context of the present disclosure PDGF encompasses PDGF-BB and PDGF-AB.

Basic fibroblast growth factor (FGF2) also known as BFGF, FGFB, HBGF-2 is a member of the fibroblast growth factor (FGF) family. FGF2 is also a regulator of cell growth and division. Both PDGF and FGF2 can be classified as mitogens in that they encourage a cell to commence cell division.

In an example, the method of the present disclosure comprises culturing a population of stem cells in a fetal bovine serum free cell culture medium comprising platelet derived growth factor (PDGF) and fibroblast growth factor 2 (FGF2), wherein the level of FGF2 is less than about 6 ng/ml. For example, the FGF2 level may be less than about 5 ng/ml, less than about 4 ng/ml, less than about 3 ng/ml, less than about 2 ng/ml, less than about 1 ng/ml. In other examples, the FGF2 level is less than about 0.9 ng/ml, less than about 0.8 ng/ml, less than about 0.7 ng/ml, less than about 0.6 ng/ml, less than about 0.5 ng/ml, less than about 0.4 ng/ml, less than about 0.3 ng/ml, less than about 0.2 ng/ml.

In another example, the level of FGF2 is between about 1 pg/ml and 100 pg/ml. In another example, the level of FGF2 is between about 5 pg/ml and 80 pg/ml. In another example, the level of FGF2 is between about 10 pg/ml and 40 pg/ml. In another example, the level of FGF2 is at least about 10 pg/ml. In another example, the level of FGF2 is at least about 11 pg/ml. In another example, the level of FGF2 is at least about 12 pg/ml. In another example, the level of FGF2 is at least about 13 pg/ml. In another example, the level of FGF2 is at least about 14 pg/ml. In another example, the level of FGF2 is at least about 15 pg/ml. In another example, the level of FGF2 is at least about 16 pg/ml. In another example, the level of FGF2 is at least about 17 pg/ml. In another example, the level of FGF2 is at least about 18 pg/ml. In another example, the level of FGF2 is at least about 19 pg/ml. In another example, the level of FGF2 is at least about 20 pg/ml. In another example, the level of FGF2 is at least about 21 pg/ml. In another example, the level of FGF2 is at least about 22 pg/ml. In another example, the level of FGF2 is at least about 23 pg/ml. In another example, the level of FGF2 is at least about 24 pg/ml. In another example, the level of FGF2 is at least about 25 pg/ml. In another example, the level of FGF2 is at least about 26 pg/ml. In another example, the level of FGF2 is at least about 27 pg/ml. In another example, the level of FGF2 is at least about 28 pg/ml. In another example, the level of FGF2 is at least about 29 pg/ml. In another example, the level of FGF2 is at least about 30 pg/ml.

In an example, the PDGF is PDGF-BB. In an example, the level of PDGF-BB is between about 1 ng/ml and 150 ng/ml. In another example, the level of PDGF-BB is between about 7.5 ng/ml and 120 ng/ml. In another example, the level of PDGF-BB is between about 15 ng/ml and 60 ng/ml. In another example, the level of PDGF-BB is at least about 10 ng/ml. In another example, the level of PDGF-BB is at least about 15 ng/ml. In another example, the level of PDGF-BB is at least about 20 ng/ml. In another example, the level of PDGF-BB is at least about 21 ng/ml. In another example, the level of PDGF-BB is at least about 22 ng/ml. In another example, the level of PDGF-BB is at least about 23 ng/ml. In another example, the level of PDGF-BB is at least about 24 ng/ml. In another example, the level of PDGF-BB is at least about 25 ng/ml. In another example, the level of PDGF-BB is at least about 26 ng/ml. In another example, the level of PDGF-BB is at least about 27 ng/ml. In another example, the level of PDGF-BB is at least about 28 ng/ml. In another example, the level of PDGF-BB is at least about 29 ng/ml. In another example, the level of PDGF-BB is at least about 30 ng/ml. In another example, the level of PDGF-BB is at least about 31 ng/ml. In another example, the level of PDGF-BB is at least about 32 ng/ml. In another example, the level of PDGF-BB is at least about 33 ng/ml. In another example, the level of PDGF-BB is at least about 34 ng/ml. In another example, the level of PDGF-BB is at least about 35 ng/ml. In another example, the level of PDGF-BB is at least about 36 ng/ml. In another example, the level of PDGF-BB is at least about 37 ng/ml. In another example, the level of PDGF-BB is at least about 38 ng/ml. In another example, the level of PDGF-BB is at least about 39 ng/ml. In another example, the level of PDGF-BB is at least about 40 ng/ml.

In another example, the PDGF is PDGF-AB. In an example, the level of PDGF-AB is between about 1 ng/ml and 150 ng/ml. In another example, the level of PDGF-AB is between about 7.5 ng/ml and 120 ng/ml. In another example, the level of PDGF-AB is between about 15 ng/ml and 60 ng/ml. In another example, the level of PDGF-AB is at least about 10 ng/ml. In another example, the level of PDGF-AB is at least about 15 ng/ml. In another example, the level of PDGF-AB is at least about 20 ng/ml. In another example, the level of PDGF-AB is at least about 21 ng/ml. In another example, the level of PDGF-AB is at least about 22 ng/ml. In another example, the level of PDGF-AB is at least about 23 ng/ml. In another example, the level of PDGF-AB is at least about 24 ng/ml. In another example, the level of PDGF-AB is at least about 25 ng/ml. In another example, the level of PDGF-AB is at least about 26 ng/ml. In another example, the level of PDGF-AB is at least about 27 ng/ml. In another example, the level of PDGF-AB is at least about 28 ng/ml. In another example, the level of PDGF-AB is at least about 29 ng/ml. In another example, the level of PDGF-AB is at least about 30 ng/ml. In another example, the level of PDGF-AB is at least about 31 ng/ml. In another example, the level of PDGF-AB is at least about 32 ng/ml. In another example, the level of PDGF-AB is at least about 33 ng/ml. In another example, the level of PDGF-AB is at least about 34 ng/ml. In another example, the level of PDGF-AB is at least about 35 ng/ml. In another example, the level of PDGF-AB is at least about 36 ng/ml. In another example, the level of PDGF-AB is at least about 37 ng/ml. In another example, the level of PDGF-AB is at least about 38 ng/ml. In another example, the level of PDGF-AB is at least about 39 ng/ml. In another example, the level of PDGF-AB is at least about 40 ng/ml.

In other examples, additional factors can be added to the cell culture medium of the present. In an example, the method of the present disclosure comprises culturing a population of stem cells in a fetal bovine serum free cell culture medium further comprising EGF. EGF is a growth factor that stimulates cell proliferation by binding to its receptor EGFR. In an example, the method of the present disclosure comprises culturing a population of stem cells in a fetal bovine serum free cell culture medium further comprising EGF. In an example, the level of EGF is between about 0.1 and 7 ng/ml. For example, the level of EGF can be at least about 5 ng/ml.

In another example, the level of EGF is between about 0.2 ng/ml and 3.2 ng/ml. In another example, the level of EGF is between about 0.4 ng/ml and 1.6 ng/ml. In another example, the level of EGF is between about 0.2 ng/ml. In another example, the level of EGF is at least about 0.3 ng/ml. In another example, the level of EGF is at least about 0.4 ng/ml. In another example, the level of EGF is at least about 0.5 ng/ml. In another example, the level of EGF is at least about 0.6 ng/ml. In another example, the level of EGF is at least about 0.7 ng/ml. In another example, the level of EGF is at least about 0.8 ng/ml. In another example, the level of EGF is at least about 0.9 ng/ml. In another example, the level of EGF is at least about 1.0 ng/ml. In another example, the level of EGF is at least about 1.1 ng/ml. In another example, the level of EGF is at least about 1.2 ng/ml. In another example, the level of EGF is at least about 1.3 ng/ml. In another example, the level of EGF is at least about 1.4 ng/ml.

In an example, the level of PDGF-BB is at least about 3.2 ng/ml, the level of EGF is at least about 0.8 ng/ml and the level of FGF2 is at least about 0.002 ng/ml. In another example, the level of PDGF-BB is at least about 9.6 ng/ml, the level of EGF is at least about 0.24 ng/ml and the level of FGF2 is at least about 0.006 ng/ml. In another example, the level of PDGF-BB is at least about 16 ng/ml, the level of EGF is at least about 0.40 ng/ml and the level of FGF2 is at least about 0.01 ng/ml. In another example, the level of PDGF-BB is at least about 32 ng/ml, the level of EGF is at least about 0.80 ng/ml and the level of FGF2 is at least about 0.01 ng/ml.

In an example, the culture medium comprises 3.2 ng/ml PDGF-BB, 0.08 ng/ml EGF and 0.002 ng/ml FGF. In another example, the culture medium comprises 9.6 ng/ml PDGF-BB, 0.24 ng/ml EGF and 0.006 ng/ml FGF. In another example, the culture medium comprises 16 ng/ml PDGF-BB, 0.4 ng/ml EGF and 0.01 ng/ml FGF. In another example, the culture medium comprises 32 ng/ml PDGF-BB, 0.8 ng/ml EGF and 0.02 ng/ml FGF. In these examples, basal medium such as Alpha MEM or StemSpan™ can be supplemented with the referenced quantity of growth factor. In an example, the culture medium comprises Alpha MEM or StemSpan™ supplemented with 32 ng/ml PDGF-BB, 0.8 ng/ml EGF and 0.02 ng/ml FGF.

In other examples, additional factors can be added to the cell culture medium of the present disclosure. For example, the cell culture media can be supplemented with one or more stimulatory factors selected from the group consisting of epidermal growth factor (EGF), 1α,25-dihydroxyvitamin D3 (1,25D), tumor necrosis factor α (TNF-α), interleukin-1β(IL-1β) and stromal derived factor 1α (SDF-1α). In another embodiment, cells may also be cultured in the presence of at least one cytokine in an amount adequate to support growth of the cells. In another embodiment, cells can be cultured in the presence of heparin or a derivative thereof. For example, the cell culture medium may contain about 50 ng/ml of heparin. In other examples, the cell culture medium contains about 60 ng/ml of heparin, about 70 ng/ml of heparin, about 80 ng/ml of heparin, about 90 ng/ml of heparin, about 100 ng/ml of heparin, about 110 ng/ml of heparin, about 110 ng/ml of heparin, about 120 ng/ml of heparin, about 130 ng/ml of heparin, about 140 ng/ml of heparin, about 150 ng/ml of heparin or a derivative thereof. In an example, the heparin derivative is a sulphate). Various forms of heparin sulphate are known in the art and include heparin sulphate 2 (HS2). HS2 can be derived from various sources including for example, the liver of male and/or female mammals. Thus, an exemplary heparin sulphate includes male liver heparin sulphate (MML HS) and female liver heparin sulphate (FML HS).

In another example, the cell culture medium of the present disclosure promotes stem cell proliferation while maintaining stem cells in an undifferentiated state. Stem cells are considered to be undifferentiated when they have not committed to a specific differentiation lineage. As discussed above, stem cells display morphological characteristics that distinguish them from differentiated cells. Furthermore, undifferentiated stem cells express genes that may be used as markers to detect differentiation status. The polypeptide products may also be used as markers to detect differentiation status. Accordingly, one of skill in the art could readily determine whether the methods of the present disclosure maintain stem cells in an undifferentiated state using routine morphological, genetic and/or proteomic analysis.

### Serum

Conventionally, stem cells are maintained in cell culture using media supplemented with at least about 10-15% v/v serum, generally fetal bovine serum (FBS), also known as fetal calf serum (FCS). The cell culture medium used in the methods of the present disclosure is a fetal bovine serum-free cell culture medium. In an embodiment, the cell culture media is supplemented with a non-fetal serum. For example, the culture media may be supplemented with a neo-natal or adult serum

In another embodiment, the cell culture medium is supplemented with human serum. In an example, the cell culture media can be supplemented with human non-fetal serum. For example, the cell culture media can be supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25% v/v human non-fetal serum.

In another example, the cell culture medium can be supplemented with human neo-natal serum. For example, the cell culture medium can be supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9% v/v human neo-natal serum. In an example, the human neo-natal serum is obtained from umbilical cord blood “cord blood”.

In another example, the cell culture medium can be supplemented with human adult serum. For example, the culture media can be supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25% v/v human adult serum.

In an example, the human adult serum is human AB serum. For example, the cell culture medium can be supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9% v/v human AB serum. In an example, the cell culture medium is supplemented with at least about 3% human AB serum.

The cell culture medium of the present disclosure may also contain known serum replacements. The serum replacement can be, for example, albumin (for example, lipid-rich albumin), transferrin, fatty acid, insulin, collagen precursor, trace element, 2-mercaptoethanol or 3′-thiol glycerol, platelet lysate, platelet-rich plasma, or those appropriately containing serum equivalents. Such a serum replacement can be prepared, for example, by a method described in International Publication WO 93/30679, and commercially available products can also be used.

In an embodiment the culture medium is serum free. In an embodiment 3D culture is FBS serum free. In an embodiment, 3D culture medium is supplemented with an above referenced xeno-free serum. In an embodiment, 3D culture medium is serum free.

### Adherent Material and Microcarriers

“Adherent material” refers to a synthetic, naturally occurring, or a combination of same, of a non-cytotoxic (i.e., biologically compatible) material having a chemical structure (e.g., charged surface exposed groups) which may retain the cells on a surface or allow cells to attach to the surface. In an example, the adherent material is a microcarrier. A “microcarrier” is a support matrix allowing for the growth of adherent cells in bioreactors. In an example, the microcarrier is a 125-250 micrometre sphere. In an example, the microcarrier has a density sufficient to be maintained in suspension with gentle stirring. Microcarriers can be made from a number of different materials including DEAE-dextran, glass, polystyrene plastic, acrylamide, collagen, and alginate. In an example, the microcarrier is porous. In another example, the microcarrier is macroporous. For example, the microcarrier can have a pore size of greater than about 50 nm. For example, the microcarrier can have a pore size of about 50 nm to about 500 nm. In other examples, the microcarrier has a pore size of about 50 nm to about 250 nm, about 50 nm to about 150 nm, about 50 nm to about 100 nm. In an example, the microcarrier is a dissolvable particle. In an example, the microcarrier comprises a glycoprotein. In an example, the glycoprotein is synthetic. In an example, the microcarrier comprises a cellulose, a glass fiber, a ceramic particle, a matrigel, an extracellular matrix component, a collagen, a poly-L-lactic acid, a dextran, an inert metal fiber, silica, glass, chitosan, or a vegetable sponge. In an example, the microcarrier comprises one or more of fibronectin, vitronectin, chondronectin, or laminin. For example, the microcarrier can be coated with one or more of fibronectin, vitronectin, chondronectin, or laminin. In other examples, the microcarrier is electrostatically charged. In an example, the microcarrier is coated with a glycoprotein. In other examples, the microcarrier is coated with collagen or gelatin. In other examples, the microcarrier is coated with collagen or vitronectin. In an example, the microcarrier is coated with vitronectin (e.g. human virtonectin). In an example, the microcarrier is coated with derivatised vitronectin (e.g. human virtonectin). In an example, the coating is synthetic. In an example, the coating is xeno-free.

In an example, the microcarrier is degradable. For example, the microcarrier can be enzymatically degradable. In an example, the microcarrier is degradable and porous. In an example, the microcarrier is degradable and macroporous. In an example, the microcarrier has a degradable core. In another example, the microcarrier has a polymer core. For example, the microcarrier can have a carbohydrate polymer core. For example, the microcarrier can have a synthetic carbohydrate polymer core. In an example, the carbohydrate polymer is linked in a calcium dependent manner. In these examples, the microcarrier core can be coated. Exemplary coatings are discussed above. For example, the microcarrier core can have a collagen or vitronectin coating.

In an example, microcarriers have a density between 0.5 to 3 g/ml. In another example, microcarriers have a density between 0.5 and 2 g/ml. In an example microcarriers have a density of about 1 g/ml.

Other examples of microcarriers are summarised by Chen et al. 2020 Biotechnology Letters, 42:1-10.

Examples of microcarriers suitable for use in the disclosure include Cultispher-G microcarrier and Corning DMC microcarriers. In an example, these microcarriers can be coated with xenofree coatings. In an example, these microcarriers are coated with a glycoprotein. For example, the microcarriers may be coated with collagen or a nectin such as vitronectin or a synthetic derivative thereof.

In an example, adherent material and microcarriers disclosed herein are degraded as part of the methods disclosed herein. Those of skill in the art will appreciate that the means for degrading the adherent material or microcarrier will be dictated by their composition. For example, adherent material or microcarriers may be degraded by enzymatic digestion. For example, vitronectin coated microcarriers can be degraded using rPectinase. In these examples, adherent material or microcarriers can be degraded by adding an enzyme to the culture medium. Other examples of suitable enzymes, depending on the nature of degradation required, include TrypLE and collagenase.

In an example, the culture medium comprises between 0.5 and 12 g/L of microcarriers. In another example, the culture medium comprises between 0.5 and 10 g/L of microcarriers. In another example, the culture medium comprises between 0.5 and 5 g/L of microcarriers. In another example, the culture medium comprises between 0.5 and 3 g/L of microcarrier. In another example, the culture medium comprises 1 g/L of microcarriers. For example, the culture medium can comprise 1 g/L of collagen coated microcarriers. In another example, the culture medium can comprise 1 g/L of vitronectin coated microcarriers.

### 3D Culture and Bioreactors

In some embodiments, culturing according to the disclosure is effected in 3D culture. For example, 3D culture can be performed in a bioreactor. Examples of such bioreactors include, but are not limited to, a plug flow bioreactor, a continuous stirred tank bioreactor and a stationary-bed bioreactor. For example, a three dimensional plug flow bioreactor (as described in U.S. Pat. No. 6,911,201) is capable of supporting the growth and prolonged maintenance of adherent cells described herein. In this bioreactor, adherent stromal cells are seeded on microcarriers discussed above, packed in columns, thereby enabling the propagation of large cell numbers in a relatively small volume. Other 3D bioreactors can be used in the present disclosure. Another example is a continuous stirred tank bioreactor. Various stirred tank bioreactors are available commercially. Those of skill in the art would appreciate that impeller position may need to be optimised. Other examples include a stationary-bed bioreactor, an air-lift bioreactor, a cell seeding perfusion bioreactor and Radial-flow perfusion bioreactor. Other bioreactors which may be used in accordance with the present disclosure are described in U.S. Pat. Nos. 6,277,151, 6,197,575, 6,139,578, 6,132,463, 5,902,741 and 5,629,186. In another example, the bioreactor is a stirred tank bioreactor. In another example, the bioreactor is a packed bed bioreactor. In an example, the packed bed bioreactor is a stirred-tank, single-use vessel. In an example, the packed bed bioreactor is the BioBLU series of vessels manufactured by Eppendorf.

In an example, cells are cultured in 3D culture for at least 5 days. In another example, cells are cultured in 3D culture for at least 6 days. In another example, cells are cultured in 3D culture for at least 7 days. In another example, cells are cultured in 3D culture for at least 8 days. In another example, cells are cultured in 3D culture for at least 9 days. In another example, cells are cultured in 3D culture for at least 10 days. In another example, cells are cultured in 3D culture for between 5 and 10 days. In another example, cells are cultured in 3D culture for between 6 and 8 days. Those of skill in the art will appreciate that cells are generally cultured to peak cell density. Time to peak cell density may be dictated by the number of cells seeded. Accordingly, in another example, cells are seeded at about 10,000 cells/ml and cultured in 3D culture for at least 6 days. In another example, cells are seeded at about 10,000 cells/ml and cultured in 3D culture for at least 7 days. In another example, cells are seeded at about 10,000 cells/ml and cultured in 3D culture for between 6 and 8 days.

In an example, between 60 and 80% of medium is replaced every 24 hours. In another example, between 65 and 75% of medium is replaced every 24 hours. In an example, about 70% of medium is replaced every 24 hours. In these examples, medium may be replaced by perfusion of medium into and out of the bioreactor. In these examples, medium is replaced from day 3 of culture in the bioreactor.

### Peak Cell Density

When performing the methods of the present disclosure, in an example, cells are cultured to peak cell density in 3D culture. For example, cells can be cultured to peak cell density in a bioreactor. In an example, culturing according to the methods of the present disclosure results in viable cell numbers plateauing after reaching peak cell density. In an example, viable cell numbers are greater than 75% 24 hours after reaching peak cell density. In another example, viable cell numbers are greater than 80% 24 hours after reaching peak cell density. In another example, viable cell numbers are greater than 85% 24 hours after reaching peak cell density. In another example, viable cell numbers are greater than 90% 24 hours after reaching peak cell density. In another example, viable cell numbers are greater than 95% 24 hours after reaching peak cell density. In an example, viable cell numbers are greater than 75% 48 hours after reaching peak cell density. In another example, viable cell numbers are greater than 80% 48 hours after reaching peak cell density. In another example, viable cell numbers are greater than 85% 48 hours after reaching peak cell density. In another example, viable cell numbers are greater than 90% 48 hours after reaching peak cell density. In another example, viable cell numbers are greater than 95% 48 hours after reaching peak cell density.

In an example, the methods of the present disclosure produce between 15 to 20 billion cells in a 50 L bioreactor. In another example, the methods of the present disclosure produce between 15 to 18 billion cells in a 50 L bioreactor. In another example, the methods of the present disclosure produce between 15 to 20 billion cells in a 50 L bioreactor, wherein the starting culture medium volume is 40 L. In another example, the methods of the present disclosure produce between 15 to 18 billion cells in a 50 L bioreactor, wherein the starting culture medium volume is 40 L. In these examples, as would be appreciated by those of skill in the art, the starting culture medium will need to be replaced over time and therefore, the total volume of culture medium used to reach the specified number of cells will be greater than 40 L.

### Compositions

The present disclosure encompasses compositions which comprise a population of mesenchymal lineage precursor or stem cells and cell culture medium, wherein the cell culture medium is serum free and comprises an adherent material, PDGF and FGF2, and wherein the mesenchymal lineage precursor or stem cells are attached to the adherent material. In an example, the adherent material is an above referenced microcarrier.

In an example, the composition may optionally be packaged in a suitable container with written instructions for a desired purpose, such as mixing of the composition with cell culture media to provide a specific concentration.

In an example, the composition is provided in a bioreactor.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

The present application claims priority from AU2020901931 filed 11 Jun. 2020, the disclosures of which are incorporated herein by reference.

### EXAMPLES

### Example 1: Medium Formulation

### Human Platelet Lysate

Human platelet lysate (hPL) is widely reported in the published stem cell literature to be a significantly more potent stimulus for mesenchymal lineage cell (MLC) growth than fetal bovine serum (FBS). Two lots of hPL from 4 different vendors were screened for their ability to stimulate the proliferation of three different lots of MLC (each derived from different donors) in a short term proliferation assay in 96 well plates. Based on this initial screen, the performance of an additional seven lots of hPL from a single vendor on one lot of MLC was examined. The results demonstrated a dose dependent stimulation of MLC in response to increasing concentrations of hPL plateauing at approximately 5% vol/vol. In accord with the published literature, at the optimal concentration of hPL, proliferation was more than 2-fold greater than that elicited by 10% FBS in the same assay (data not shown). Based on these data, hPL was used as a replacement for FBS in three replicate BioR runs in a Millipore Mobius 50 L CellReady bioreactor using the same MLC bank as used in previous runs with culture medium comprising FBS. These three runs were performed using Solohill Collagen-coated microcarriers (collagen-coated polystyrene microcarriers).

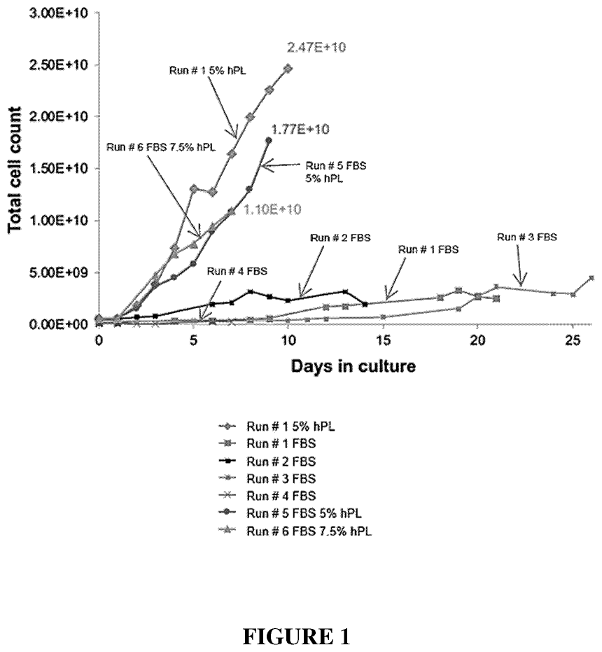

Shown in FIG. 1 are runs conducted in medium supplemented with 5% and at 7.5% vol/vol hPL. These runs demonstrated a marked increase in the rate of cell proliferation with MLC reaching cell numbers between 11 billion (day 7) to 24.7 billion (day 10) on the days the runs were terminated. The three runs generated peak numbers of 10.7 billion (day 11), 11.2 billion (day 9) and 19.9 billion (day 10) MLC on the days stated in parentheses. Collectively, these data indicate that medium composition is key to ensuring a high yielding bioreactor (BioR) process for manufacture of MLC. FBS is clearly a very poor stimulus for MLC growth on microcarriers in the Millipore Mobius 50 L SUB while hPL represents a much more potent stimulus in accord with its effects on MLC proliferation in 2-D.

### V2.2 Medium

However, a significant concern with the use of hPL for MLC manufacture at a commercial scale is the availability of sufficient quantities. A thorough supply chain analysis is lacking to estimate supply available for cell therapy product manufacture. In addition, lot sizes of hPL are currently small and require pooling from multiple donors. Finally, there is currently no consensus as to the most appropriate pathogen depletion strategies required to eliminate potential for transmission of human pathogens. For these reasons V2.2 medium was developed. This medium is free of animal components and contains an targeted combination of recombinant mitogens for proliferation of MLC. V2.2 medium comprises basal medium supplemented with FGF2, PDGF and EGF along with other requirements for cell growth. Thus, V2.2 medium eliminates both the supply constraints and the safety issues that limit the use of hPL.

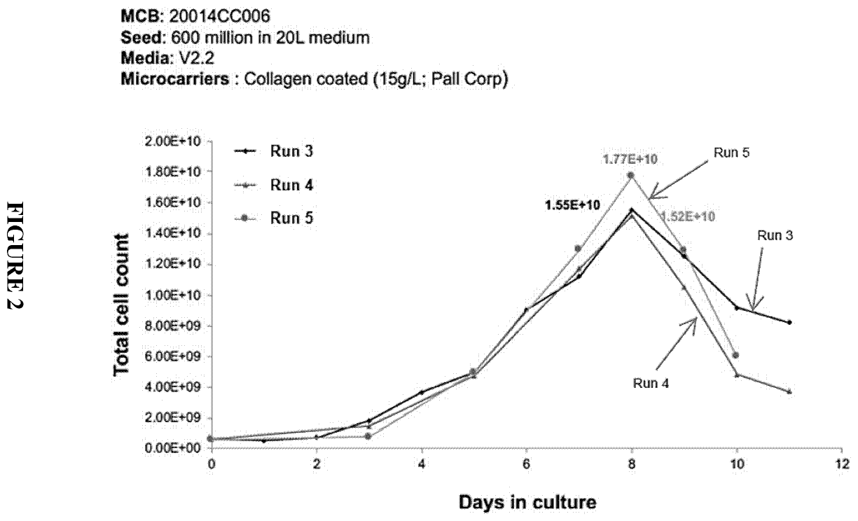

Testing of V2.2 was again conducted with the same MLC cell bank as used in previous experiments to eliminate the MLC as a variable and to allow cross comparison of the data with other changing variables. Once again, cells were seeded at 600 million in the Millipore Mobius 50 L Cell Ready bioreactor containing Solohill collagen-coated microcarriers at 15 g/L. As shown in FIG. 2, in 3 replicate runs V2.2 supported very robust proliferation of the MLC which expanded rapidly reaching peak densities in all 3 replicates at day 8, peak cell density at this time varying between 15.2 to 17.7 billion cells. Thereafter there was a precipitous and problematic decline in cell numbers over the next 24-48 hours.

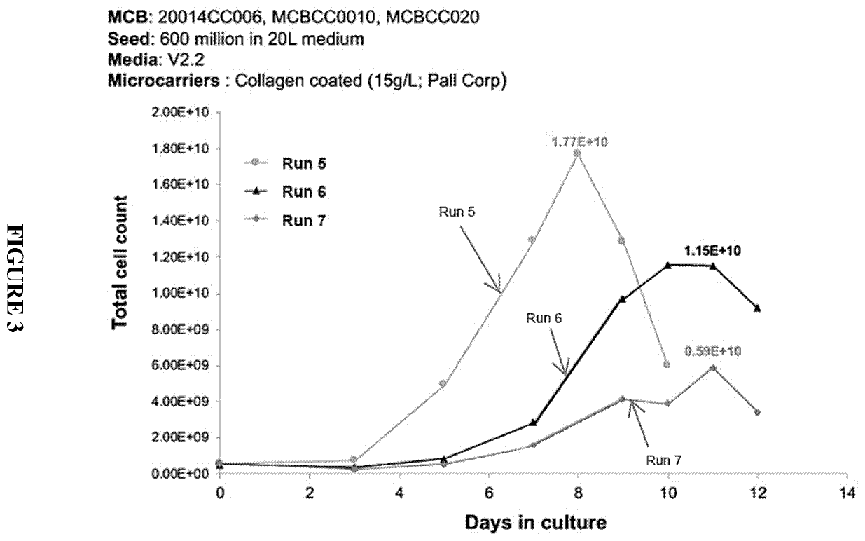

When tested using master cell bank (MCB) MCB006 in parallel with MLC from two additional donors, MCB010 and MCB020, V2.2 supported proliferation of all three. However, the rapid decline in cell numbers previously observed following reaching peak cell density for MCB006 was also observed for the two additional donors tested (FIG. 3).

In summary, V2.2 demonstrated the ability to support robust proliferation of MLC from multiple donors in the Millipore Mobius 50 L CellReady bioreactor when grown on collagen coated microcarriers. However, after reaching peak cell density in each run, cell numbers rapidly declined. Measurement of lactate and ammonium levels at the time of the crash did not demonstrate toxic levels nor were glucose levels limiting or the levels of recombinant cytokines. In additional runs which attempted to prevent this crash in cell numbers by increasing the frequency of medium exchange around the time of the decline failed to prevent the crash. However, as shown in Example 3 below, by changing the microcarrier and the bioreactor design, a stable plateau at peak cell density was achieved.

### Example 2: Optimisation of Seed Preparation and Seeding of Microcarriers

The following set of experiments highlight other components of BioR Process Development using the V2.2 medium directed at optimizing seeding of the microcarriers and preparation of the seed itself. As noted above all experiments in the Millipore Mobius 50 L CellReady bioreactor in FBS, hPL and V2.2 up to this point were performed using a seed of 600 million cells. To determine the optimal seeding density for seeding the Solohill collagen-coated microcarriers in V2.2 medium, experiments were conducted in Corning 125 mL spinner flasks.

The effect of seeding cell density on the final number of MLC recovered at harvest on Day 7 was examined (data not shown). Spinners (in triplicate) containing 100 mL V2.2 and collagen coated microcarriers at 15 g/L were seeded with between 100,000 to 2 million MLC, equivalent to seeding densities of 1,000, 2,000, 5,000, 8,000, 10,000 and 20,000 MLC/mL. The number of cells harvested at the endpoint progressively increased between 0.1 to 0.5 million seeded cells reaching a plateau at ˜0.8 million cells. Based on these data the number of cells seeded per mL in all subsequent experiments at small and large scale was 10,000/mL.

As noted above, the optimal seed density per mL was determined to be 10,000 MLC at donor cell bank stage. For a 40 L BioR volume this equates to 400 million cells. To determine the optimal media exchange/harvest times required to reproducibly generate this number of cells from the contents of a single MCB vial in one CF10 cell factory in V2.2 medium, preliminary experiments were conducted with MCBs from two different donors which consistently produce a high or low yield. The contents of single vials of each donor were seeded into 3 CF10 cell factories and subjected to the following V2.2 media exchange/time of harvest protocols to identify which condition(s) yielded >400 million cells at harvest:

Day 3 media exchange/Day 5 harvest

Day 4 media exchange/Day 6 harvest

Day 3 & 5 media exchange/Day 7 harvest

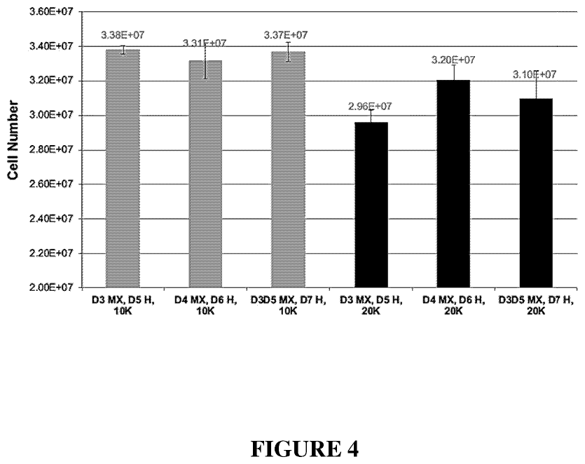

Group B (Day 4 media exchange/Day 6 harvest) (D4MX/D6H) emerged as the minimum condition required to generate this cell number from both the slow and high yielding MCBs. Cells from the D4MX/D6H strategy and from the other two groups were then tested in spinners, seeding at both 10,000/mL and at 20,000/mL in 100 mL of V2.2+collagen coated MC at 15 g/L. Notably, the media exchange/harvest protocol appeared to have no influence on the yield of MLC in the spinners as shown in FIG. 4.

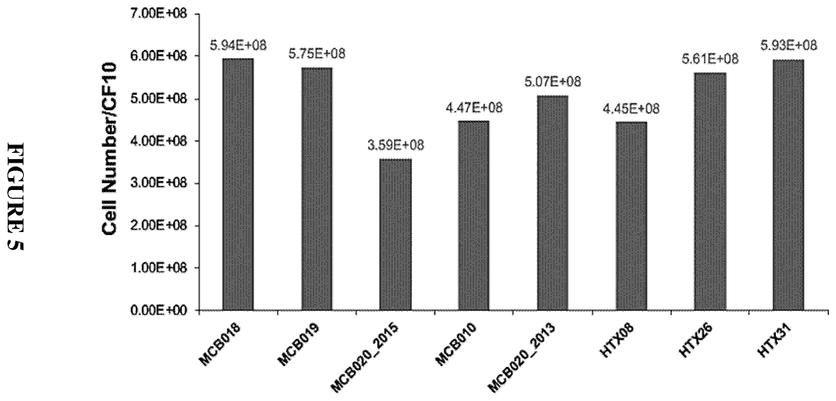

Shown in FIG. 5 is the application of this D4MX/D6H protocol to MCBs from 8 different donors. In all but one MCB was the minimum target number of 400 million cells obtained. Based on these data, the D4MX/D6H protocol for seed preparation was adopted for the remaining examples.

### Example 3: Microcarrier Choice and Bioreactor Design

In the previous Examples all runs, both spinners and at the 50 L scale, had been conducted with the Solohill collagen-coated microcarriers. One issue with the use of these polystyrene carriers is the need to remove them at the end of the run during the downstream processing phase of the process. In addition, the need to use 15 g/L which equates to 0.75 kg in a 50 L bioreactor impacts on cost of goods.

Given that MLCs bind well to collagen, Cultispher-G microcarriers were evaluated. Cultispher-G microcarriers are a macroporous microcarrier composed of porcine collagen from Percell Biolytica. Aside from the larger surface area provided by these carriers, another advantage is that at the end of the run they can be removed simply by enzymatic digestion of the gelatin. In preliminary experiments it was determined that TryPLE at a 2× final concentration was sufficient to completely dissolve the carriers and that this concentration and duration of digestion had no adverse effects on the viability of MLCs.

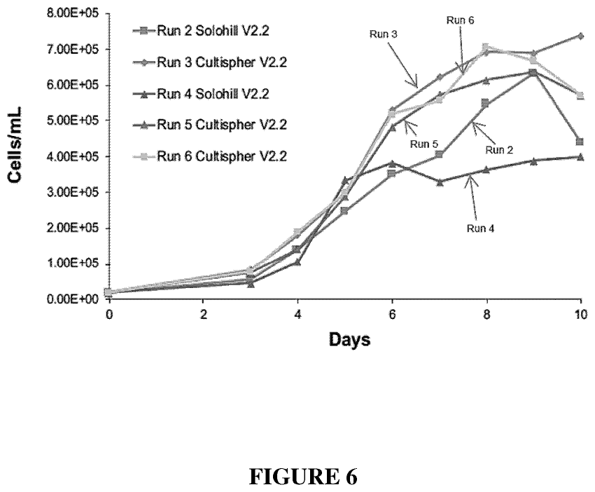

In addition, additional bioreactor platform, the BioBLU series of vessels manufactured by Eppendorf, was evaluated. A series of experiments using the Cultispher-G microcarriers at both the spinner and intermediate scale (BioBLU 3c) were conducted. A representative experiment is shown in FIG. 6. MCB019 was seeded at 10,000/mL at full vessel volume (2.5 L) into triplicate BioBLU 3c vessels in V2.2 containing either Cultispher-G microcarriers (1 g/L) or Solohill collagen coated microcarriers (15 g/L). At day 4 and everyday thereafter, 70% of the volume of the medium was replaced with fresh V2.2 (batch process). The combination of the Cultispher-G microcarriers and BioBLU vessel resulted in steady increase in MLC number/mL reaching a stable plateau at ˜day 7 with peak numbers of 738,000, 637,000 and 708,000/mL for the three runs. In contrast, one of the runs conducted with the Solohill carriers achieved peak cell density of only 382,000/mL at day 5 with a stable plateau thereafter while in the second run, cell numbers progressively increased to day 9 (635,000/mL) but exhibited a significant drop in numbers to 441,000/mL over the next 24 hours. These data suggest that use of glycoprotein coated micro carriers such as the Cultispher-G microcarriers and/or the BioBLU vessel design may prevent the dramatic crash in cell numbers previously seen when using the combination of Solohill microcarriers in the Millipore Mobius BioR vessels. This impression was further supported by the outcome of additional experiments using the BioBLU 3c (data not shown) which confirmed that use of the combination of the Cultispher-G microcarriers/BioBLU vessel design was consistently associated with the generation of a stable plateau in cell number following achievement of peak cell density and appeared to obviate the significant decline in cell numbers observed with the Solohill microcarrier/Millipore Mobius combination.

To test this hypothesis at full scale, 3 runs were conducted in which Cultispher-G (1 g/L) carriers were tested in the BioBLU 50c vessels (40 L scale) using for comparison, the same MLC bank MCB006 as used in the earlier PD runs with the Solohill carriers and the Mobius 50 L bioreactor. These 3 runs were conducted in V2.2 and cells were seeded at 10,000/mL. Feeding was by a batch process in which 70% of the medium was replaced from day 4 of the run until its termination.

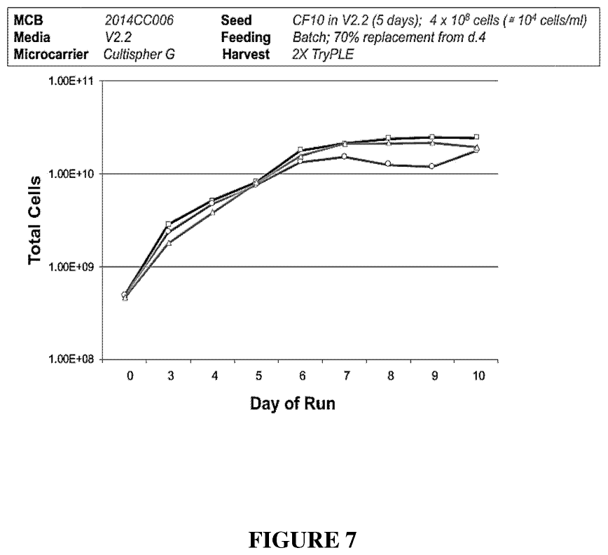

As shown in FIG. 7, the use of the combination of Cultispher-G microcarriers with the BioBLU 50c resulted in steady, exponential increases in cell number in all 3 replicate runs reaching a peak with a mean of 19.1 billion cells at day 7. Most significantly, and in complete agreement with data at the BioBLU 3c scale (FIG. 6), there was a stable plateau of cell number in all 3 runs until the end of the runs at day 10, in marked contrast to the dramatic decline in cell numbers that characterizes previous runs performed with the Millipore Mobius 50 L/Solohill collagen coated microcarrier combination (FIG. 2 and FIG. 3).

Finally, a fully synthetic, animal component free, dissolvable microcarrier (DMC) formulation was compared with those of Cultispher-G and the Solohill collagen-coated polystyrene microcarriers for performance of these carriers at the spinner scale. The DMC formulations comprised Corning DMC particles coated with either xeno-free collagen or synthetic, flexible vitronectin-based peptide substrate Synthemax. The DMC formulations were assessed to address the concern that if the process were to be conducted with the Cultispher-G microcarriers which comprise porcine collagen, despite their obvious advantages, the process as a whole would obviously not be free of animal proteins.

All groups were performed using triplicate spinners in V2.2 and cells from MCB MB006 were seeded at 10,000 cells/mL. Media replacement was by a batch process with a 70% medium replacement from day 4 through day 7.

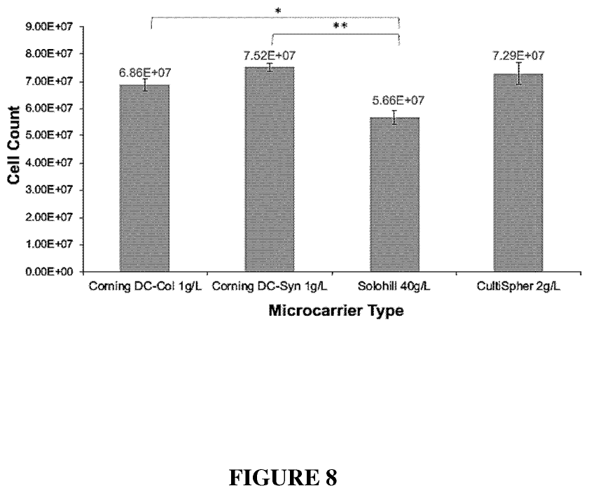

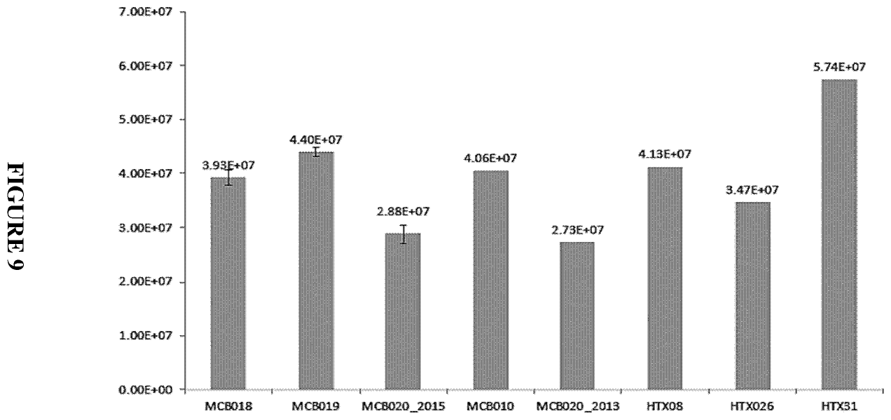

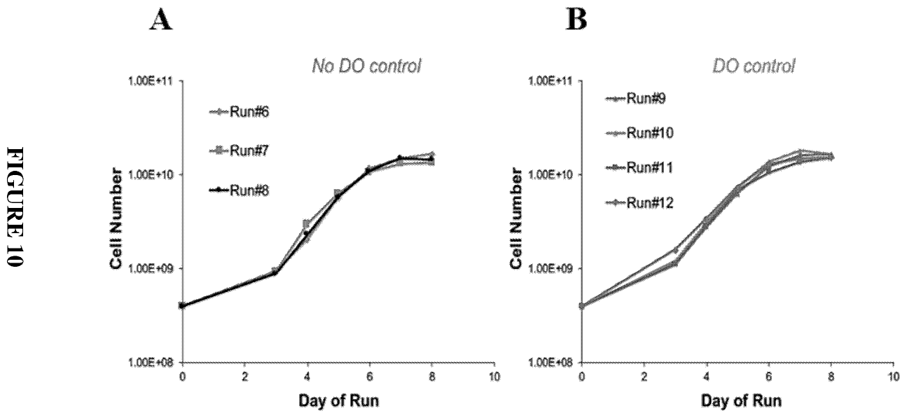

At the time of harvest on day 7, MLC reached densities of between 570,000/mL (Solohill) and 730,000/mL (Corning DMC-Synthemax; Cultispher-G). Both the Collagen-coated Corning DMC and the Corning-DMC coated with synthetic vitronectin peptide, Synthemax demonstrated significantly higher yields than that obtained using the Solohill carriers. There was no significant difference between yields obtained by either of the Corning-DMC-collagen or Corning DMC-Synthemax formulations vs. that supported by Cultispher-G microcarriers (FIG. 8). Comparison of yields obtained in spinner flasks with the Corning DMC-Synthemax across a range of different cell banks demonstrated the expected variation that is typically observed between MLC donors (FIG. 9). Importantly all exhibit high levels of proliferation. For example, MCB019 at 440,000 cells/mL extrapolates to −17 billion cells at a 40 L scale in the BioBLU 50c, a number close to that actually obtained in 50c runs with this cMLC cell bank (see FIG. 10). Based on the performance of the Corning DMC-Synthemax demonstrated in these spinner flask cultures, their performance was next evaluated in the BioBLU 50c.

A perfusion-based methodology for medium exchange in the BioBLU 50c was also optimised. This methodology avoided the disadvantages of the batch feeding process and like the batch process, replaces 70% of the volume in the vessel every 24 hours from day 3 (data not shown).

The protocol for harvesting cells at the end of a run using the Corning DMC-Synthemax had also been optimised and included Corning's recommended protocol for microcarrier dissolution together with brief use of TryPLE to break up cell-cell contacts. The resulting cell suspension was collected into a bag (Flex Concepts) and passed into the kSep 400 for washing and concentration prior to cryopreservation of the product. Seven runs were conducted which incorporated both the perfusion based medium exchange strategy and downstream harvesting methodology together with the kSep washing and concentration step. The runs were conducted with a single MCB, MCB019. In 4 runs dissolved oxygen (DO) was maintained while in the remaining 3 it was not controlled. Seed was prepared as described above, seeded at 10,000 cells/mL into 40 L of V2.2 medium containing Corning Synthemax DMC at 1 g/L. Analytes and cell counts were measured daily. A 1 L harvest was performed on day 7 and a full harvest of the remaining cells on day 8.

FIG. 10 illustrates highly reproducible yields of MLC obtained on the Corning DMC-Synthemax at the 40 L scale in the BioBLU 50c. At a mean of 1.43+0.09 billion the yield in the absence of DO control is not significantly different from that obtained with DO control, at 1.56+0.86 (p=0.176).

In view of the above, it was noted that glycoprotein coated particles such as Corning DMC-Synthemax and use of the BioBLU bioreactor were, unexpectedly, advantageous in their own right, in particular in relation to halting rapid decline in cell numbers after cultures reached peak cell density. Accordingly, these data support use of microcarriers that are coated with a nectin peptide such as Synthemax in 3D culture of MLPSCs.

The above Examples also identified the combination of the Eppendorf BioBLU 50c bioreactor, Corning DMC-Synthemax microparticles and V2.2 medium as an animal component-free BioR process that supports reproducibly high levels of MLC yields at day 7.

### Example 4: Extended Characterization of the MSC Products Generated in the V2.2/2-D Downscale Process

To investigate whether MLC propagated as outlined above exhibit appropriate identity, purity and potency markers, the cells harvested from each run were cryopreserved and upon thaw subjected to a comprehensive range of bioanalytics to determine any effects on critical quality attributes (CQAs) of the cells. The extended characterization analytics included post-thaw viability and recovery, cell size, proliferation capacity, identity, purity, cytokine secretion and immunomodulatory potential as measured by the ability to inhibit T-cell proliferation.

### Post-Thaw Viability and Recovery

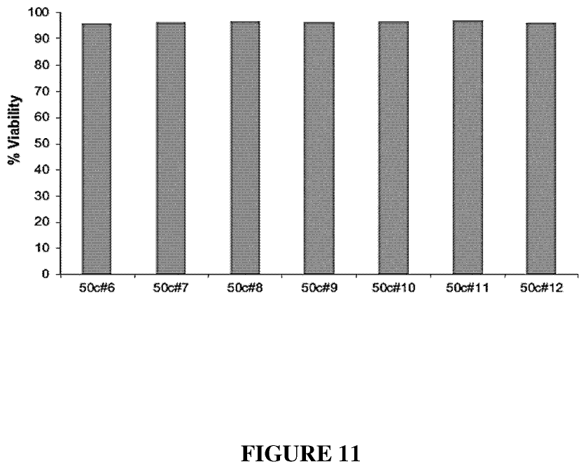

Cryopreserved MLC from each of the 7 test samples were thawed according to RD.SOP.04.06 and immediate post thaw viability measured by Trypan Blue exclusion according to the methodology described in PR-031. Post thaw viability was extremely consistent between all samples ranging between 96-97% with no significant difference+DO regulation (FIG. 11).

### Post-Thaw MSC Diameter

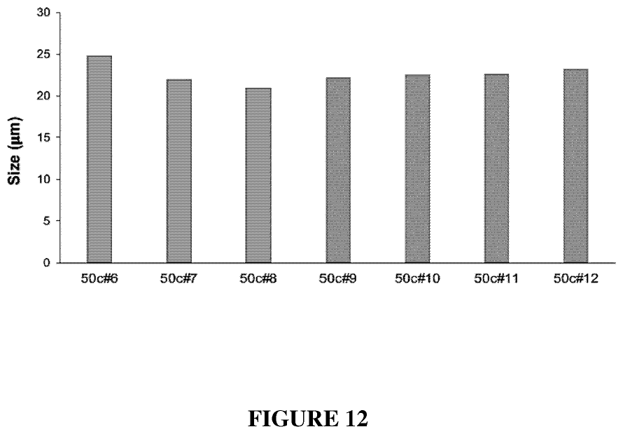

MLC size in suspension following thaw of each of the 8 cryopreserved test articles was estimated by flow cytometry using microbeads of known sizes as reference standards. Microbead sizes were determined by the manufacturers using scanning electron microscopy and National Institute of Standards and Technology (NIST) traceable particles. Microbeads from Spherotec were used as references for the following cell diameters: 2 μm, 3 μm, 5 μm, 7 μm, 10 μm and 14.5 μm, while microbeads from Bangs Laboratories were used for cell sizes 20 μm, 25 μm, 30 μm and larger. A standard curve was generated from a range of reference standards (typically spanning 5-30 μm) by plotting microbead size against its forward scatter (FS) signal determined by flow cytometry (linear or log peak maximum, FS-median). Thus, the sizes of ceMSC test samples were determined from the standard curve using the FS-median of the sample. Testing was performed on freshly thawed samples. As shown in FIG. 12, all 7 test articles generated on Corning DMC-Synthemax carriers in V2.2 in the BioBLU 50c exhibited a very similar cell diameter ranging from 21 μm (run #8) to 24.8 μm. Those generated in the absence of DO control exhibit an average cell diameter of 22.6+1.63 μm while those produced with DO control demonstrated a mean diameter of 22.6+0.36 μm (p=0.477).

### Post-Thaw Proliferation Capacity

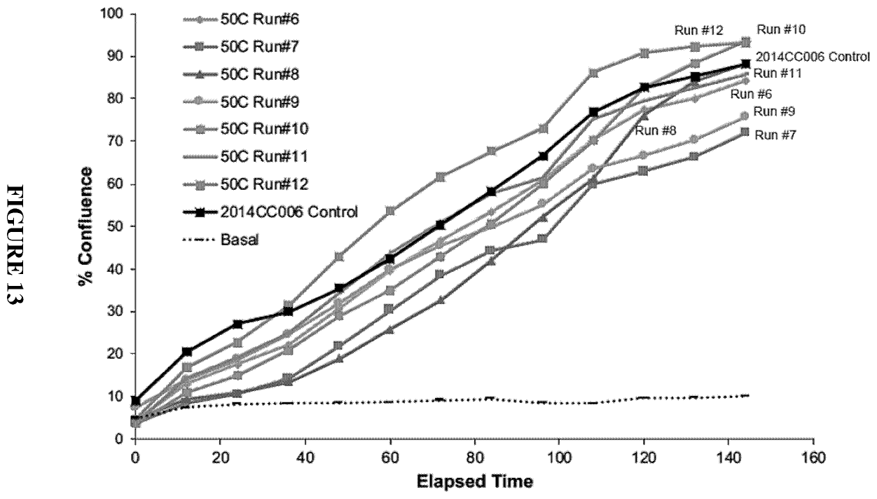

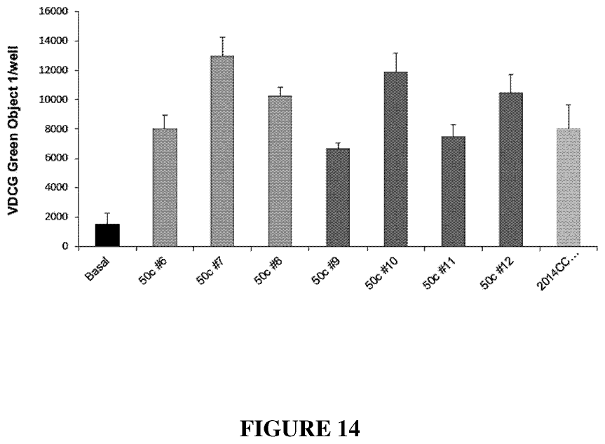

Analysis of the proliferation kinetics of MLC in culture following thaw from cryopreservation provides a quantitative measurement of MSC functionality beyond the immediate measurement of cell viability. Each of the 7 lots of MLC generated in the bioreactor runs were seeded in triplicate at 2,000 cells per well in serum-supplemented growth medium in 96-well plates. A mesenchymal lineage cell (MLC) lot expanded to passage 5 and seeded in 10% fetal bovine serum (FBS) in Minimum Essential Medium alpha (αMEM) was used as a suitability control. This suitability control has reliably demonstrated proliferative activity in this assay; its activity in each experimental run therefore serves to indicate the suitability of essential reagents, materials and equipment used in the test procedure. In addition, as a negative control, each sample, including the suitability control, was seeded in serum-free (basal) medium. Cultures were incubated for 140-146 hours inside the IncuCyte® ZOOM live-imaging microscope (Essen BioScience) fitted into a humidified cell culture incubator set at 5% CO₂, 37±2° C. and simultaneously imaged every 6 hours. At the end of the incubation time, Vybrant™ DyeCycle™ Green (Invitrogen) was added prior to scanning the plates in the IncuCyte Zoom using the green filter set. The number of nuclei/well present in each condition tested at the end of the incubation time and the percent confluency across the duration of the assay were determined after applying the integrated algorithms to mask the nuclei stained with the reagent or the area occupied by the cells, respectively. As demonstrated in FIG. 13, all 7 of the BioR products demonstrated proliferation kinetics very similar to the suitability control 2014CC006 grown in the current 2-D/FBS process and reached levels of confluence at the day 6 endpoint of the assay ranging from 72% (Run #7) to 93.6% (Run #10). There was no significant difference between the level of confluence achieved at day 6 in the presence or absence of DO control (p=0.212). In accord with this, use of Vybrant™ DyeCycle™ Green to measure the number of cells per well (FIG. 14) similarly demonstrated that the number of cells per well at day 6 did not vary significantly between the DO and no DO control arms (p=0.257).

### Flow Cytometric Analysis of MLC Identity and Purity

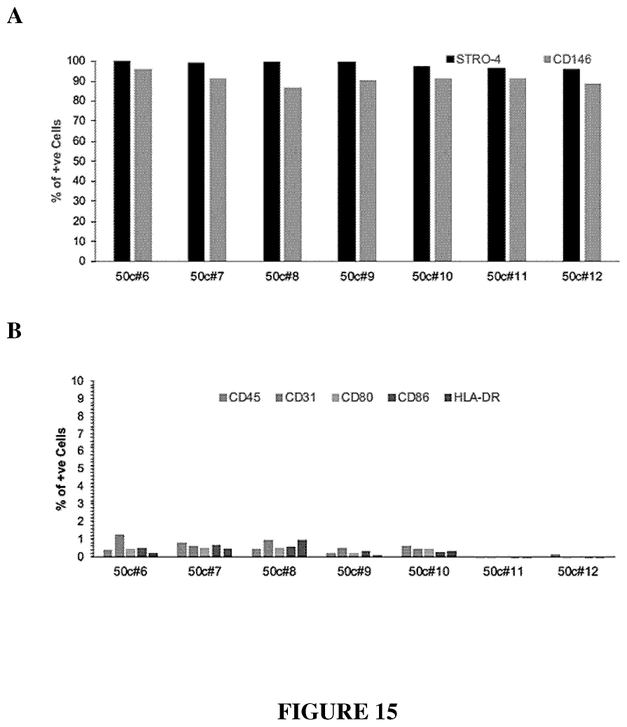

Freshly-thawed test samples were incubated with monoclonal antibodies directed against human CD146, STRO-4 (as markers of MLC identity) and with antibodies to CD45, CD31, CD80, CD86 and HLA-DR (as markers of MLC purity) and with non-binding, isotype matched negative control antibodies. The antibodies used were directly conjugated with the fluorochrome R-phycoerythrin (PE). DAPI was used to distinguish between live and dead cells. For each of the purity markers (CD80 CD86, HLA-DR), positive control cell lines were run in parallel with the ceMSC samples to confirm system suitability of the assay. Samples were analysed by flow cytometry. Following exclusion of debris based on light scatter properties and dead cells by DAPI staining, the expression level of each marker was determined by comparison of the fluorescence of the antibody-stained sample with its isotype negative control. Shown in FIG. 15 are the results of these analyses. All test articles from the 7 BioR runs expressed the purity marker CD146 at levels >86.8% and STRO-4 at between 96-99.9% and between 99.76-99.9%. For each of the 5 purity markers analysed, staining did not exceed 1.3% in any one sample and for the majority was below 1%.

### Example 5: Functional Activity of MLC

Key attributes underlying the multimodal mechanisms of action of MLC in a range of therapeutic indications is their ability to secrete a diverse repertoire of paracrine activities that, collectively, mediate the reparative and immunomodulatory functions of the cells. The secretion of several proangiogenic factors by the seven lots of MLC was examined as a measure of the actions of MLC in stimulating tissue repair through the promotion of new blood vessel development. As a measure of their immunomodulatory properties, the capacity of the cells to inhibit T-cell proliferation was also assessed.

### SDF-1α

Mesenchymal lineage cells secrete robust levels of multiple growth factors well documented to play causal roles in the formation of new blood vessels. These include SDF-1α, VEGF-A and angiopoietin-1 (ANGPT1). After thawing, the various test articles were seeded in culture at equal viable cell densities and allowed to generate conditioned media under serum-free conditions. After collection of the conditioned media, each of the proteins of interest were quantified by means of ELISA (R&D Systems).

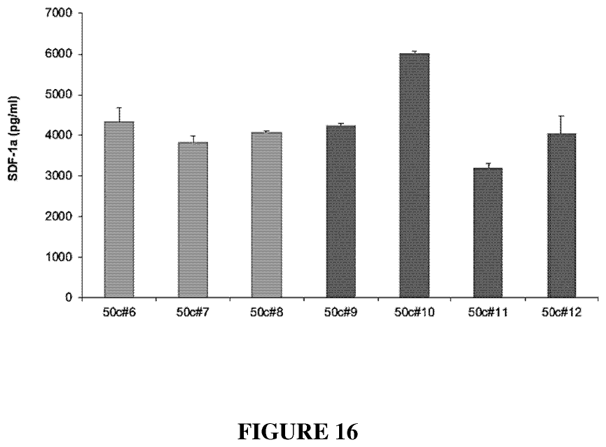

SDF-1α levels in the conditioned medium varied between the 7 MLC test articles lots ranging from approximately 3198 pg/mL to 6022 pg/mL (mean 4246+802 pg/mL) (FIG. 16). At 4069+203 pg/mL and 4379+1027 pg/mL, respectively, the mean levels of SDF-1α secreted in the absence vs. presence of DO maintenance were not significantly different (p=0.34).

Measurement of the bioactivity of SDF-1α in the same test article conditioned medium samples used for the ELISA measurement of SDF-1α concentration was also undertaken. For the SDF-1α bioassay, a cell line (U937) expressing high levels of CXCR4 was seeded into trans-well inserts and serum-free MLC conditioned media was placed in the lower well of 24-well plates. Cells were allowed to migrate for 3 hours to MLC conditioned media or recombinant human SDF-1α. Serum-free basal media that had not been conditioned and contained no measurable levels of SDF-1α was used as a negative control and recombinant human SDF-1α at 3 ng/ml was used as the system suitability assay control. Following the specified time-point of 3 hrs, the cells which had migrated to the bottom chamber were collected and mixed with CountBright Absolute Counting Beads (Invitrogen, C36950) and the numbers of migrated cells were quantitated using flow cytometry against a standard number of count beads added to each tube.

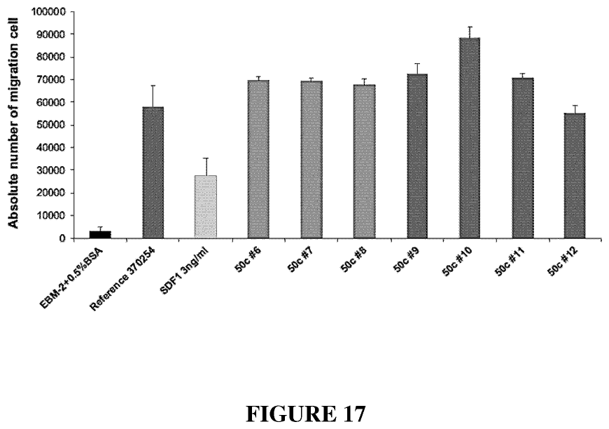

Shown in FIG. 17 are data from the migration assay. The observed levels of migration of the U937 cells to the various samples of MLC conditioned medium very closely mirror the levels of SDF-1α measured in the corresponding samples by ELISA. The average level of migration observed across all 7 samples (70,610+9067 cells) was close to that observed for MLC manufactured in a 2-D/FBS process which was used as a suitability control for these assays.

### VEGF-A

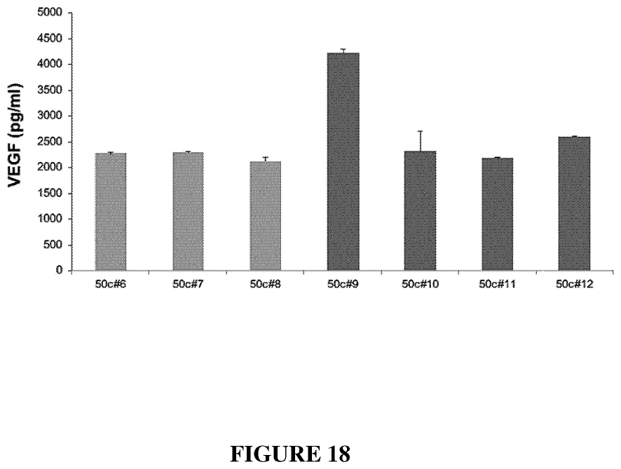

The level of VEGF in the conditioned medium samples was similarly measured by ELISA. Aside from one outlier, Run #9 which secreted VEGF at a level of 4204 pg/mL, the levels of VEGF secreted by MLC generated in the remaining 6 runs were extremely consistent with a mean of 2290+148 pg/mL (FIG. 18).

### ANGPT1

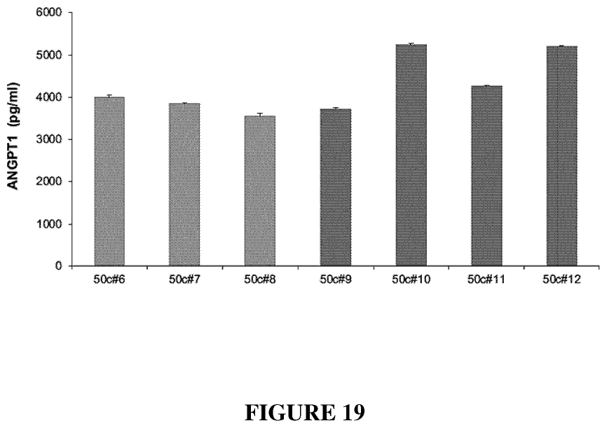

The level of ANGPT1 in the conditioned medium showed moderate variation from run to run with a mean level of secretion across all 7 runs of 4269+672 pg/mL (FIG. 19). For the 3 runs conducted in the absence of DO control, the ANGPT levels showed minimal variation (mean 3794+187 pg/mL) but the runs conducted with control of DO, the levels were somewhat more variable (4625+684) but despite these differences, the mean levels of ANGPT1 secreted in the absence vs. presence of DO maintenance were not significantly different (p=0.073; not significant at p<0.05).

### Example 6: Immunomodulatory Activity

The MLC generated in each of the seven bioreactor runs were assessed for the ability to suppress activated T-cell proliferation as a direct measure of their immunosuppressive capability and cell quality. Briefly, for this assay PBMC were stimulated with CD3 and CD28 antibodies and co-cultured with MLC at various ratios (1:5, 1:10 & 1:20) and activated T-cell proliferation was measured by EdU incorporation and multicolor flow cytometry. Controls for this assay include unstimulated PBMC alone, stimulated PBMC alone and an MLC lot manufactured in FBS as described above (suitability control).

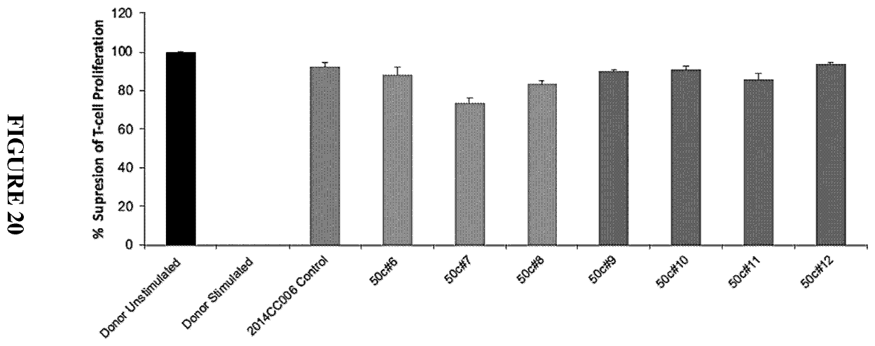

As shown in FIG. 20, 7/7 MLC test articles generated in V2.2 on Corning DMC-Synthemax in the BioBLU 50c bioreactor demonstrated very potent capacities to inhibit T cell proliferation at the 1:10 ratio with 3/7 inhibiting proliferation by >90%, a level very close to that of the suitability control which inhibited to the level of 92%. For the remaining four BioR-generated samples, the level of inhibition achieved was still very high and ranged from 73-88%.

### Example 7: Summary

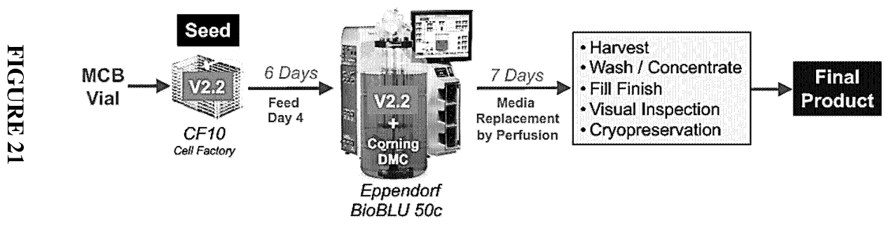

Shown in FIG. 21 is a schematic of the bioreactor process that represents the culmination of the work summarised in the present disclosure. Beginning with a single vial of the master cell bank (MCB), this is transferred to a single CF10 cell factory and used to generate the required 400 million seed for 6 days under the conditions described above. The harvested seed is then transferred to the BioBLU 50c containing 40 L of V2.2 and Corning DMC-Synthemax at 1 g/L. MLC are grown for a further 7 days in the vessel (for a total campaign time of 13 days) with medium replacement by perfusion beginning on day 3, replacing 70% of the volume of V2.2 every 24 hours. A total of 160 L of V2.2 medium is used per BioBLU 50c run. Harvesting of the cells occurs in the vessel and follows settling of the carriers and removal of 35 L of spent media. This is then followed by the addition of EDTA and pectinase (for dissolution of the Corning DMC-Synthemax) and 2×TrypLE for 30 minutes after which the contents of the vessel are transferred by peristaltic pump via a bag (FlexConcepts) to a kSep400 for washing and concentration. Washed and concentrated product is then filled, finished and visually inspected before being cryopreserved.

1. A method of culturing mesenchymal lineage precursor or stem cells in a three dimensional culture, the method comprising culturing a population of mesenchymal lineage precursor or stem cells on an adherent material in a cell culture medium, wherein the mesenchymal lineage precursor or stem cells are attached to the adherent material and, wherein the cell culture medium is animal serum free.

2. The method of claim 1, wherein the cell culture medium comprises platelet derived growth factor (PDGF) and fibroblast growth factor 2 (FGF2).

3. The method of claim 2, wherein the cell culture medium also comprises EGF.

4. The method according to any one of claims 1 to 3, wherein the mesenchymal lineage precursor or stem cells are cultured in a bioreactor.

5. The method according to any one of claims 1 to 4, wherein the adherent material is a microcarrier.

6. The method of claim 5, wherein the microcarrier has a degradable core.

7. The method of claim 5 or claim 6, wherein the microcarrier has a carbohydrate polymer or glycoprotein core.

8. The method according to any one of claims 1 to 7, wherein the adherent material or microcarrier is coated with a glycoprotein.

9. The method of claim 7 or claim 8, wherein the glycoprotein is a collagen or vitronectin.

10. The method of claim 9, wherein the vitronectin is human vitronectin or a synthetic mimetic thereof.

11. The method according to any one of claims 7 to 10, wherein the glycoprotein is synthetic.

12. The method of claim 11, wherein the carbohydrate polymer is linked in a calcium dependent manner.

13. The method according to any one of claims 1 to 12, wherein the culture medium comprises 0.5 g/L to 5 g/L of microcarrier.

14. The method according to any one of claims 5 to 13, wherein the microcarrier is a porous microcarrier.

15. The method of claim 14, wherein the microcarrier is a macroporous microcarrier.

16. The method according to any one of claims 1 to 15, wherein the culture medium is free of animal components.

17. The method according to any one of claims 1 to 16, wherein about 70% of medium is replaced every 24 hours of culture.

18. The method according to any one of claims 1 to 17, further comprising dissociating the mesenchymal lineage precursor or stem cells from the adherent material by contacting them with a dissociating agent.

19. The method of claim 17 or claim 18, further comprising vibrating the adherent material for a period of time at a frequency and amplitude sufficient to release the mesenchymal lineage precursor or stem cells from the adherent material.

20. The method according to any one of claims 1 to 19, further comprising degrading the adherent material.

21. The method according to claim 20, wherein the adherent material is degraded by adding an enzyme to the culture medium.

22. The method according to any one of claims 1 to 21, wherein mesenchymal lineage precursor or stem cells are seeded at between 5,000 and 20,000 cells/ml.

23. The method according to any one of claims 1 to 21, wherein mesenchymal lineage precursor or stem cells are seeded at 10,000 cells/ml.

24. The method according to any one of claims 1 to 23, wherein mesenchymal lineage precursor or stem cells have been culture expanded from a master cell bank.

25. The method of claim 24, wherein the mesenchymal lineage precursor or stem cells have been culture expanded from a master cell bank in a two dimensional culture format.

26. The method according to any one of claims 1 to 25, further comprising recovering the cells from the culture medium and cryopreserving the recovered cells.

27. The method of claim 26, wherein the recovered cells are washed and concentrated prior to cryopreservation.

28. The method according to any one of claims 1 to 27, wherein the mesenchymal lineage precursor or stem cells are cultured in a three dimensional culture for at least 6 days, preferably between 5 and 8 days, more preferably 7 days.

29. The method according to any one of claims 4 to 28, wherein the bioreactor is a stirred tank and/or packed bed bioreactor.

30. A composition comprising a population of mesenchymal lineage precursor or stem cells and cell culture medium, wherein the cell culture medium is animal serum free and comprises an adherent material, PDGF and FGF2, and wherein the mesenchymal lineage precursor or stem cells are attached to the adherent material.

31. The composition of claim 30, wherein the adherent material is as defined in any one of claims 5 to 15.

32. The method according to any one of claims 1 to 29 or the composition according to claim 30 or 31, wherein the mesenchymal lineage precursor or stem cells are mesenchymal precursor cells or mesenchymal stem cells.

33. The method according to any one of claim 1 to 29 or 32 or, the composition according to claim 30 or 31, wherein the PDGF in the culture medium is PDGF-BB.

34. The method according to any one of claim 1 to 29 or 32 or 33 or, the composition according to any one of claims 30 to 32, wherein the culture medium:
comprises between 3.0 ng/ml and 120 ng/ml of PDGF-BB;
comprises between 2 pg/ml and 6 ng/ml of FGF2;
comprises less than 0.8 ng/ml of FGF2;
further comprises EGF.

35. The method according to any one of claims 1 to 29 or 32 to 34 or, the composition according to any one of claims 30 to 34, wherein the culture medium further comprises between 0.08 ng/ml and 7 ng/ml of EGF.

36. The method according to any one of claims 1 to 29 or 32 to 35 or, the composition according to any one of claims 30 to 35, wherein the culture medium comprises alpha-minimal essential medium or fetal bovine serum free expansion medium.

37. The method according to any one of claims 1 to 29, 32 to 36 or, the composition according to any one of claims 30 to 36, wherein the culture medium maintains the stem cells in an undifferentiated state.

38. A method of culturing stem cells in a bioreactor, the method comprising culturing a population of mesenchymal lineage precursor or stem cells in a bioreactor comprising cell culture medium, wherein the cell culture medium is animal serum free and comprises platelet derived growth factor (PDGF) and fibroblast growth factor 2 (FGF2) and optionally EGF.
